(19) United States
(12) Patent Application Publication
(10) Pub. No.: US 2022/0395537 A1
(43) Pub. Date: Dec. 15, 2022

(54) METHODS OF STEM CELL CULTURE FOR OBTAINING PRODUCTS, AND IMPLEMENTATIONS THEREOF

(71) Applicant: Pandorum Technologies Private Limited, Bangalore (IN)

(72) Inventors: Tuhin BHOWMICK, New York, NY (US); Arun CHANDRU, Bangalore (IN); Deepthi MENON, Bangalore (IN); Shivaram SELVAM, Hyderabad (IN); Midhun BEN THOMAS, Bangalore (IN); Wenson David RAJAN, Bangalore (IN)

(21) Appl. No.: 17/578,442

(22) Filed: Jan. 18, 2022

Related U.S. Application Data
(63) Continuation of application No. PCT/IN2020/050622, filed on Jul. 18, 2020.

(30) Foreign Application Priority Data
Jul. 18, 2019 (IN) 201941029039
Jul. 18, 2019 (IN) 201941029040
Jul. 18, 2019 (IN) 201941029041
Jul. 18, 2019 (IN) 201941029042

(51) Int. Cl. A61K 35/30; C12N 5/00; C12N 5/079

(52) U.S. Cl. CPC A61K 35/30; C12N 5/0018; C12N 5/0621; C12N 2500/95

(57) ABSTRACT

The present disclosure discloses methods for culturing stem cells in three-dimensional methods. Said method is either a spheroid-based method or a microcarrier-based method. The process as described herein leads to the expansion of the stem cells to obtain an expanded population of the stem cells, and a stem cell derived-conditioned medium. The present disclosure also discloses an expanded population of the stem cells, and a stem cell derived-conditioned medium obtained from the process as described herein. Further, an exosome preparation obtained from the stem cell derived-conditioned medium is also disclosed herein. The present disclosure also discloses a composition comprising an expanded population of the stem cells, or a stem cell derived-conditioned medium, or an exosome preparation, or combinations thereof. Methods of treatment using the composition as described herein is also disclosed in the present disclosure.

### CROSS-REFERENCE TO RELATED APPLICATIONS

This application is a Continuation of International Application No. PCT/IN2020/050622, filed on Jul. 18, 2020, which claims priority to Indian Application No. 201941029039, filed on Jul. 18, 2019, Indian Application No. 201941029040, filed on Jul. 18, 2019, Indian Application No. 201941029041, filed on Jul. 18, 2019, and Indian Application No. 201941029042, filed on Jul. 18, 2019. All applications are hereby incorporated by reference in their entirety.

### FIELD OF INVENTION

The present disclosure broadly relates to the field of in-vitro cell culture techniques, and particularly refers to methods of culturing stem cells in order to obtain expanded stem cell population along stem cell derived-conditioned medium. The present disclosure also discloses a process for obtaining expanded corneal stromal stem cell population, and the expanded corneal stromal stem cell population and a corneal stromal stem cell derived-conditioned medium. The present disclosure also discloses the secreted cell modulators such as exosomes and protein factors from the conditioned medium (stem cell derived-conditioned medium and corneal stromal stem cell derived-conditioned medium) which will be used for the regenerative treatment & inflammatory diseases of various tissues/organ such as the cornea, lung, liver, kidney, heart, pancreas, and brain or combination thereof (such as multi-organ failure).

### BACKGROUND OF INVENTION

Mesenchymal Stem cells (MSCs) have the capacity to divide, self-renew or differentiate into different cell types. The ability of the mesenchymal stem cells to differentiate into different cell types make them an ideal source of cells for regenerative therapy for the management of various conditions like lung infections, neurological disorders, Parkinson's disease etc. However, one of the major challenges with the modality of the treatment is the requirement of large number of mesenchymal stem cells per dosage so to have an enhanced therapeutic effect. Conventionally, MSCs isolated from various tissues are normally cultured as monolayers in a 2D system in tissue culture flasks. Although 2D system is easy to handle, however, it is time consuming & labor intensive when large volume of cells is required to be harvested. For instance, an average patient with 80 kg body weight requires a billion MSCs to facilitate a positive therapeutic intervention which can be monumental to scale-up via a 2D culture system. Further, 2D culture system induces changes in morphology and gene expression of cultured cells which can behave significantly differently compared to cells in native 3D tissues. Accordingly, the current methods of culturing stem cells in 2D culture system are not amenable to scale up the production of MSCs. Hence, the limitations of 2D system have paved the way for using 3D culture system for the large-scale production of mesenchymal stem cells. In the 3D culture system, cells are grown in a 3D microenvironment that offer multidirectional cellular interactions simulating physiological conditions in-vivo. When cells are grown in 3D culture systems, cells also induce the formation of aggregates or spheroids within matrix or the culture medium. In the 3D spheroid culture system, cells are allowed to aggregate and naturally self-assemble to form spheroid or microtissues. Although it is demonstrated that 3D spheroid culture systems enhance the properties of the MSCs and facilitates their interaction under native forces allowing them to secrete extra-cellular matrix proteins, however, the large-scale production of the MSCs and production of MSCs-derived exosomes in clinically relevant doses in not achieved.

Accordingly, there exists a need in the art to provide an improved and cost-effective method for large-scale production of MSCs and to amplify the yield of MSCs-derived exosomes. The amplified MSC-derived exosomes can then be further used for various clinical applications in a safe and reproducible way.

### SUMMARY OF THE INVENTION

In an aspect of the present disclosure, there is provided a process for culturing stem cells to obtain a population of expanded stem cells, said method comprising: (a) obtaining a population of stem cells; and (b) culturing the stem cells of step (a) in either a spheroid-based system or a microcarrier-based system, to obtain a population of expanded stem cells, and a stem cell derived-conditioned medium.

In another aspect of the present disclosure, there is provided a process for obtaining a stem cell derived-conditioned medium, said process comprising: (a) obtaining a population of stem cells; and (b) culturing the stem cells of step (a) in either a spheroid-based system or a microcarrier-based system, to obtain a population of expanded stem cells, and a stem cell derived-conditioned medium.

In another aspect of the present disclosure, there is provided a process for culturing stem cells, to obtain a population of expanded stem cells, said process comprising: (a) obtaining a population of stem cells; (b) obtaining microcarriers comprising crosslinked alginate core and crosslinked gelatin surface; (c) suspending the microcarriers in a culture medium, to obtain a suspension; (d) seeding the suspension with the population of stem cells of step (a); (e) culturing the stem cells of step (d) in a culture medium to obtain a population of expanded stem cells adhered to the microcarriers, and a stem cell derived-conditioned medium; and (f) dissolving the microcarriers of step (e) by contacting the microcarriers with a dissolution buffer comprising sodium chloride and trisodium citrate, to obtain a population of expanded stem cell.

In another aspect of the present disclosure, there is provided a process for culturing stem cells, to obtain a population of expanded stem cells, said process comprising: (a) obtaining a population of stem cells; (b) pelleting the stem cells of step (a), to obtain a stem cell pellet; (c) resuspending the stem cell pellet in a culture medium comprising basal medium, to obtain a stem cell suspension; (d) obtaining stem cell spheroids from the stem cell suspension obtained in step (c), wherein the stem cell spheroids are having a density of stem cells in a range of 600-10,000 cells per spheroid; and (e) culturing the stem cell spheroids of step (d) in a culture medium comprising basal medium to obtain a population of expanded stem cells, and a stem cell derived-conditioned medium.

In another aspect of the present disclosure, there is provided a stem cell derived-conditioned medium obtained by the process as described herein.

In another aspect of the present disclosure, there is provided an expanded mesenchymal stem cell population obtained by the process as described herein.

In another aspect of the present disclosure, there is provided an expanded stem cell population obtained by the process as described herein.

In another aspect of the present disclosure, there is provided a composition comprising the stem cell derived-conditioned medium as described herein.

In another aspect of the present disclosure, there is provided a composition comprising the expanded stem cell population as described herein.

In another aspect of the present disclosure, there is provided an exosome preparation obtained by a process comprising: (a) harvesting the stem cell derived-conditioned medium as described herein, to obtain a secretome; (b) centrifuging the secretome, to obtain a pellet; (c) dissolving the pellet in a low serum xeno free medium, to obtain a crude solution; (d) performing density gradient ultracentrifugation with the crude solution, to obtain a fraction comprising exosomes; and (e) purifying the fraction comprising the exosomes by size exclusion chromatography, to obtain an exosome preparation.

In another aspect of the present disclosure, there is provided a composition comprising at least two components selected from the group consisting of: (a) the expanded stem cell population as described herein, (b) the stem cell derived-conditioned medium as described herein, and (e) the exosome preparation obtained by the process as described herein.

In another aspect of the present disclosure, there is provided a process for isolating and culturing corneal limbal stem cells, to obtain an expanded corneal stromal stem cell population, said process comprising: (a) obtaining a limbal ring tissue from a human donor cornea; (b) mincing the tissue, to obtain tissue fragments; (c) suspending the fragments in an incomplete medium, to obtain a suspension; (d) subjecting the fragments to digestion in the presence of at least one type of collagenase enzyme at a concentration range of 5-20 IU/μl with respect to the suspension, to obtain digested explants; (e) culturing the digested explants in a complete medium comprising 1-10% human platelet lysate for a period of 10-14 days, to obtain a population of corneal limbal stem cells; and (f) passaging the corneal limbal stem cells of step (e) for a period of 10-14 days, to obtain an expanded corneal stromal stem cell population, and a corneal stromal stem cell derived-conditioned medium.

In another aspect of the present disclosure, there is provided a method for treating a condition selected from the group consisting of corneal disorders, liver fibrosis, and hyper-inflammatory conditions, said method comprising: (a) obtaining the exosomes as described herein; and (b) administering the exosomes to a subject for treating the condition.

In another aspect of the present disclosure, there is provided a method for treating a condition selected from the group consisting of corneal disorders, liver fibrosis, and hyper-inflammatory conditions, said method comprising: (a) obtaining the stem cell derived-conditioned medium as described herein; and (b) administering a therapeutically effective amount of the conditioned medium to a subject for treating the condition.

In another aspect of the present disclosure, there is provided a method for treating a condition selected from the group consisting of corneal disorders, liver fibrosis, and hyper-inflammatory conditions, said method comprising: (a) obtaining the expanded stem cell population as described herein; and (b) administering a therapeutically effective amount of the expanded stem cell population to a subject for treating the condition.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the characterization of CSSCs isolated by the xenofree protocols as disclosed in the present disclosure; comparison of expression of CSSC specific markers (CD90/CD73/CD105) confirms the protocol employing Liberase (LIB) for digestion and MEM media for culture as optimal for the xenofree culture of CSSCs; Scale bar: 100 μm, in accordance with an embodiment of the present disclosure.

FIG. 2 depicts the characterization of CSSCs isolated by LIB_MEM protocol in accordance with an embodiment of the present disclosure.

FIG. 3 depicts the characterization of hBM-MSCs (RoosterBio Inc.); Key: Lane 1: D200: Donor #200; Lane 2: D227: Donor 227; Lane 3: D257: Donor 257. Scale bar: 100 μm, in accordance with an embodiment of the present disclosure.

FIG. 4 depicts the schematic depiction of core crosslinked alginate beads (crosslinked with divalent or trivalent ions and their combinations thereof) possessing glutaraldehyde crosslinked gelatin to promote cell attachment, in accordance with an embodiment of the present disclosure.

FIG. 5 depicts the flowchart depicting the steps involved in the preparation of alginate microbeads crosslinked with Ca²⁺/Ba²⁺ ions with a cell adhesive gelatin crosslinked surface, in accordance with an embodiment of the present disclosure.

FIG. 6A depicts the phase contrast image of the microbeads, B) depicts the size distribution of the microbeads and C) depicts the circularity distribution profile. Scale bar 250 mm, in accordance with an embodiment of the present disclosure.

FIG. 7 depicts the Cell adherence and viability on fabricated Alg/Gel microbeads. a) Phase contrast image and b) Live dead assay on BM-MSC adhered microbeads 24 h after cell loading in static conditions. c) Phase contrast image of BM-MSCs and d) Live dead assay on BM-MSC adhered microbeads after static loading (24 h) and 72 h in dynamic condition. Scale bar: 200 mm, in accordance with an embodiment of the present disclosure.

FIG. 8 depicts the Live dead assay performed on a) PS beads, b) RCP beads and c) Alg/Gel microbeads. Dotted line represents outline of bead surface. Scale bar: 100 mm, in accordance with an embodiment of the present disclosure.

FIG. 9 depicts the Immunostaining for aSMA on a) PS beads, b) RCP beads and c) Alg/Gel microbeads. Lower aSMA expression (GREEN) was observed in Alg/Gel and RCP microcarriers compared to PS beads. (d-f) represents CD90 (RED) stem cell marker expression of cultured cells on PS, RCP and Alg/Gel microbeads. Dotted line represents outline of bead surface. Scale bar: 100 mm, in accordance with an embodiment of the present disclosure.

FIG. 10 depicts the microbeads of the present disclosure (Alg/Gel microbeads) with cells treated with dissolution buffer. a) at 0 mins, b) after 1 min, c) after 7 mins and d) cell viability assay using trypan blue demonstrating 80% viability. Scale bar: 200 mm, in accordance with an embodiment of the present disclosure.

FIG. 11 depicts the scheme depicting the generation of scalable MSC spheroids, in accordance with an embodiment of the present disclosure.

FIG. 12 depicts the A. Phase-contrast images taken 24 hr and 48 h after seeding the cells in the hanging drop with or without methylcellulose. B. Confocal images of viability staining from the spheroid from day 2 and 5 showing the minimal cell death in the spheroids cultured in both +methylcellulose and −methylcellulose. Scale bar: 200 μm, in accordance with an embodiment of the present disclosure.

FIG. 13 depicts the (A) Confocal images of viability staining from the spheroid at a seeding density of 1500 cells from day 4 showing minimal cell death in the spheroids cultured in both +methylcellulose and −methylcellulose (hanging drop method). Scale bar: 50 μm. (B) Confocal images of viability staining from the spheroid at an initial seeding density of 10,000 cells from day 4 showing minimal cell death in the spheroids cultured in both +methylcellulose and −methylcellulose (hanging drop method). Scale bar: 200 μm, in accordance with an embodiment of the present disclosure.

FIG. 14A depicts a schematic summary of the experiment executed for the hanging drop-spinner flask culture of hBM-MSC spheroids. FIG. 14B depicts phase-contrast microscopy images of spheroids taken on day 0 of static hanging drop culture, day 3 and day 7 in the spinner flask culture showing the compactness of the spheroids were well maintained during the culture period. FIG. 14C depicts Live-Dead staining performed on day 3 and day 7 in the spinner culture. FIG. 14D depicts whole-spheroid immunofluorescence staining of CD90 (MSC marker) performed on day 7 of the spinner flask culture. FIG. 14E depicts whole-spheroid immunofluorescence staining of alpha-SMA performed on day 7 of the spinner flask culture. Scale bar: 200 μm, in accordance with an embodiment of the present disclosure.

FIG. 15A depicts the Schematic summary of the experiment executed for the direct-spinner flask culture of hBM-MSC spheroids. FIG. 15B depicts phase-contrast microscopy images of spheroids taken on day 2, 3 and 5 post-seeding in the spinner flask. FIG. 15C depicts Live-Dead staining on spheroids performed on day 2 and day. Scale bar: 200 μm, in accordance with an embodiment of the present disclosure.

FIG. 16 depicts the scheme for isolation of exosomes by Iodixanol density gradient ultracentrifugation, in accordance with an embodiment of the present disclosure.

FIG. 17 depicts the secretory cytokine profile of BMMSCs and CSSCs in 2D culture. (A) BMMSCs secrete more IL-6 than CSSCs; (B) CSSCs secrete more HGF than BMMSCs. (C) CSSCs secrete less VEGF compared to all three BMMSC donors, in accordance with an embodiment of the present disclosure.

FIG. 18 depicts the (A) CSSCs secrete more HGF than BMMSCs. CSSC priming (10% CSSC-CM & 25% CSSC-CM) modestly improved HGF secretion in BMMSC Donor #200. (B) BMMSCs secrete more IL-6 than CSSCs. CSSC priming (10% CSSC-CM & 25% CSSC-CM) decreased the IL-6 secretion by BMMSCs. Since it is only one donor, data is not conclusive. (C) CSSCs secrete less VEGF compared to all three BMMSC donors. (D) Nerve Growth factor (NGF) and soluble Fms Related Receptor Tyrosine Kinase 1 (sFLT1) were detected in CSSC secretome while BMMSC-secretome from three donors (ID #200, #227 and #257) did not express detectable levels of the proteins (by western blot). Priming of BMMSC Donor #200 with CSSC-CM induced the secretion of NGF and sFLT1 in the secretome at both 10% and 25% supplementation, in accordance with an embodiment of the present disclosure.

FIG. 19A depicts the comparison of exosome population isolated by Single step ultracentrifugation (UC_Step1), 30% sucrose cushion and iodixanol gradient ultracentrifugation protocols: FIGS. 19B-19C demonstrate the heterogeneity of the exosome particle size obtained in each method of purification. FIG. 19B shows that single step UC purification of exosomes results in isolation of particles in the range of 50-170 nm and 30% sucrose cushion gives us particles in the range of 60-150 nm. FIG. 19C shows that iodixanol gives us a tighter range of 30-130 nm, in accordance with an embodiment of the present disclosure.

FIG. 20 depicts the Particle concentration of fraction 9 (F9): 1.8×10¹⁰/ml) (A and B); C. Median particle diameter in nm ranged between 100-150 nm; D. Avg. size distribution of F9: 28-133 nm. Particle size distribution and particle number were determined by NTA. Particles were detected at 11 different positions of the cell and averaged. Each sample was run in 3 technical replicates. E. Exosomes (fraction 9) isolated from hBM-MSCs were positive for typical exosome markers including CD63, CD9, CD81, ALIX and TSG101, in accordance with an embodiment of the present disclosure.

FIG. 21 depicts the Transmission Electron Microscopy (TEM) images of exosomes isolated by iodixanol density gradient ultracentrifugation. Lower magnification of representative images is shown in (A) and the respective magnified image (marked in yellow box) is shown in (B). Scale bars (0.2 um (E), and 200 nm (F)). The TEM images shows exosomes in the expected size range of about 150-250 nm range and complements the NTA data, in accordance with an embodiment of the present disclosure.

FIG. 22 depicts the Exosome size distribution and cargo characterization post size exclusion chromatography. (A-D) All fractions up to F7 were run on NTA. From F5, no particles were detected and only alternate fractions were run thereon. (E) Particle concentration per fraction (Fraction 9 was diluted into two fractions (2+3). (F) Flow cytometry analysis of fraction 2 and 3 from captocore purification identified 75% and 54% of the exosome population in fraction 2 and fraction 3 to be CD81/CD9 positive, respectively. (G) Western blot analysis of exosome markers CD81, CD9, CD63, ALIX and TSG101 in captocore purified fraction 9, in accordance with an embodiment of the present disclosure.

FIG. 23A depicts the Size distribution analysis of exosomes purified from BMMSCs by 30% cushion-based sucrose density method using Nano Tracking Analysis (NTA). FIG. 23A shows a representative image of a histogram. FIG. 23B shows averaged data from 3 independent readings of size distribution. FIG. 23C shows the total yield of exosomes from 30% sucrose cushion ultracentrifugation determined by NTA. FIG. 23D shows a Western blot analysis for exosome marker CD9. FIGS. 23E-23F show Protein samples from secretome and exosome preparation were separated on a 12% SDS PAGE gel and antibody against CD9 was used to identify exosomes. CD9 was present both in secretome and exosome samples showing expected size of 24-27 Kda and the control samples were negative. FIG. 23E shows Transmission Electron Microscopy (TEM) images of exosomes isolated by 30% sucrose method. Lower magnification of representative images. FIG. 23F shows the respective magnified image (marked in yellow box). Scale bars (0.2 um (FIG. 23E), and 200 nm (FIG. 23F)). The TEM images shows exosomes in the expected size range of about 150-250 nm range and complements the NTA data, in accordance with an embodiment of the present disclosure.

FIGS. 24A-24F depict the Size distribution analysis of exosomes purified from CSSCs by 30% sucrose cushion density (30% SUC) based ultracentrifugation (FIGS. 24A-24C) and iodixanol density gradient ultracentrifugation (IDX Fraction 9 (IDX-F9)) method (FIGS. 24C-24D) using Nano Tracking Analysis (NTA). A representative image of histogram is shown in FIG. 24A and FIG. 24D for 30% SUC and IDX-F9, respectively. The averaged data from 3 independent readings of size distribution are presented in FIG. 24B & FIG. 24E for 30% SUC and IDX-F9, respectively. FIG. 24C shows the total yield of exosomes from 30% SUC and IDX-F9 respectively determined by NTA. FIG. 24F shows Western blot analysis for exosome marker CD9 for 30% SUC and IDX-F9. Protein samples from secretome and exosome preparation were separated on a 12% SDS PAGE gel and antibody against CD9 was used to identify exosomes. CD9 was present both in secretome and exosome samples showing expected size of 24-27 Kda and the control samples were negative, in accordance with an embodiment of the present disclosure.

FIG. 25 depicts the reproducibility of the exosome purification protocol (iodixanol density gradient ultracentrifugation) as disclosed in the present disclosure, in accordance with an embodiment of the present disclosure.

FIG. 26 depicts the comparison of purity of exosomes purified by three methods (i) single step ultracentrifugation (UC_step1), (ii) s\30% sucrose cushion (iii) iodixanol gradient UC (IDX). (A) Sucrose cushion and iodixanol gradient methods gave comparable purity and low levels of VEGF compared to UC_Step 1 (single step ultracentrifugation) while retaining therapeutic factors such as HGF (B), in accordance with an embodiment of the present disclosure.

FIG. 27 depicts the comparison of scalability of CSSC-CM primed MSCs versus CSSC in clinical applications, in accordance with an embodiment of the present disclosure.

FIG. 28 depicts the characterization of adipose derived mesenchymal stem cells cultured as per the method in accordance with an embodiment of the present disclosure.

FIG. 29 depicts the four strategies used for isolating and culturing of corneal limbal stem cells to obtain an expanded population of corneal stromal stem cells, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

For the purposes of the present document, the term “corneal limbal stem cells” refers to the population of stem cells which reside in the corneal limbal stem cell niche. The corneal limbal stem cell is referred to population of stem cells represented majorly by corneal stromal stem cells (CSSC), and limbal epithelial stem cells (LESC).

The term “stem cell” intends to cover all types of stem cells that are well-known in the art. As part of several implementations of the present disclosure, the disclosure discloses mesenchymal stem cell, and corneal limbal stem cells. The term “a population of expanded stem cells” denotes the population of stem cells which has increased number of cells as compared to the population of stem cells obtained initially for culturing. The culturing process does not differentiate the cells, it just increases the number of cells manifolds. Similarly, the term “a population of expanded corneal stem cells” denotes the population of corneal stem cells which has increased number of cells as compared to the population of corneal stem cells obtained initially for culturing. The culturing process does not differentiate the cells, it just increases the number of cells manifolds. The term “naive” or “un-primed” refers to the stem cells which are not primed with any factors including CSSC-CM.

The terms “a population of expanded mesenchymal stem cells refers to the population of mesenchymal stem cells which has an increased number of cells as compared to the population of mesenchymal stem cells obtained initially for culturing. The culturing process does not differentiate the cells, it just increases the number of cells manifolds. The term “three-dimensional” or “3D” refers to a system of culturing the cells in-vitro in which the biological cells are allowed to grow and interact with their surroundings in all the three dimensions. The term “two-dimensional” or “2D” refers to the method of culturing the cells on a surface by which the biological cells are able to interact with their surroundings in two dimensions. The term “spheroid-based system” refers to the process of culturing stem cells (MSC) in a three-dimensional manner by formation of spheroids according to the method as described in the present disclosure. The term “microcarrier-based system” refers to the process of culturing mesenchymal stem cells (MSC) in a three-dimensional manner by the formation of alginate-gelatin (Alg/Gel) microcarriers or microbeads according to the method as described in the present disclosure. The term “microcarriers” and “microbeads” are used interchangeably, it refers to the alginate-gelatin (Alg/Gel) microcarriers or microbeads as described in the present disclosure.

The term “stem cell derived-conditioned medium” denotes the medium obtained after the growth of any kind of stem cells. The term “mesenchymal stem cell derived-conditioned medium or “MSC-CM” refers to the medium obtained after the growth of the MSC. The term “corneal stromal stem cell derived-conditioned medium” refers to the conditioned medium obtained after the growth/enrichment of corneal stromal stem cells. The conditioned medium thus obtained comprises secreted cell modulators and multiple factors critical for tissue regeneration. The conditioned medium thus obtained also comprises secretome, and exosomes which needs to be purified from the conditioned medium before being able to apply for therapeutic purposes. The process for obtaining expanded stem cells The process for obtaining expanded MSC as described herein also leads to the formation of MSC-CM, therefore, it can be said that a single process leads to the procurement of a population of expanded MSC as well as of MSC-CM. The term “exosomes” refers to the type of an extracellular vesicle that contain constituents (in terms of protein, DNA, and RNA) of the biological cells that secretes them. The exosomes obtained from the conditioned medium as described herein is used for therapeutic purposes.

The term “corneal stromal stem cell derived-conditioned medium or “CSSC-CM” refers to the medium in which corneal stromal stem cells (CSSC) are grown. The CSSC-CM as described herein is obtained by culturing of CSSC in a manner known in the art or by culturing of CSSC as per the method disclosed herein.

The term “xeno-free” as described in the present disclosure refers to the process as described herein which is free of any product which is derived from non-human animal. The method being xeno-free is an important advantage because of its plausibility of clinical application. The term “scalable” refers to the ability to increase the production output manifolds. The term “subject” refers to a human subject who is suffering from the conditions as mentioned in the present disclosure. The term “therapeutically effective amount” refers to the amount of a composition which is required for treating the conditions of a subject.

The term “culture medium” refers to the medium in which the MSC is cultured. The culture medium comprises MSC basal medium, and the MSC basal medium is used as per the MSC which is being cultured. The MSC basal medium as mentioned in the present disclosure was commercially procured. For the purposes of the present disclosure, RoosterBio xenofree media was used for BMMSCs.

The term “low serum xeno free medium” refers to the standard xeno free medium which is low on the serum level which is commercially available for the purposes of culturing MSC. It can be contemplated that a person skilled in the art can use any such medium for the purposes of the present disclosure.

As per the present disclosure, the products derived from the cell culture methods as disclosed herein comprises the expanded (cultured) stem cell population which can be mesenchymal stem cells or corneal stromal stem cells, conditioned medium derived from the respective type of stem cells. The conditioned medium is further used to purify cell-derived products such as secretome, exosome, and other extracellular matrix (ECM) components like biopolymers. The cell-derived components are further used for the methods of treatment as disclosed herein and for various regenerative purposes. The process as described in the present disclosure is an in-vitro process, i.e. taking place in an artificially created environment outside of the living being.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

In order to overcome the existing problems in the field, the present disclosure discloses methods of culturing mesenchymal stem cells, wherein the method is a xeno-free method and a scalable method in order to provide ease of commercialization of the process. Further, the present disclosure discloses the three-dimensional based methods which can either be spheroid-based or microcarrier-based method to obtain expanded mesenchymal stem cell population and a mesenchymal stem cell derived-conditioned medium. Therefore, the present disclosure discloses the method to achieve higher protein production which can thus be translated to higher amounts of cell secreted factors that can be isolated for example, secretome, exosome, and other cell derived proteins. The present disclosure also discloses the secreted cell modulators such as exosomes and protein factors from the conditioned medium which can be used for the regenerative treatment & inflammatory diseases of various tissues/organ such as the cornea, lung, liver, kidney, heart, pancreas, and brain or combination thereof (such as multi-organ failure). The present disclosure discloses an approach to increase the production of expanded mesenchymal stem cells so as to translate it in a clinical level. Also, the present disclosure discloses the conditioned medium obtained by culturing of the mesenchymal stem cells, and the cell secreted factors like exosomes which can be obtained in multiple folds as compared to the techniques known in the art. The present disclosure also discloses a method of isolating and purifying exosomes from the conditioned medium for using the same for regenerative purposes.

One of the central objectives of the present disclosure is to be able to develop a scalable process for the production of biomaterials consisting of extra cellular matrix activated by secreted cell modulators. For achieving the objectives, the present disclosure discloses the process of xeno-free cell culturing using three-dimensional approaches that will generate scalable quantities of cell modulators of therapeutic benefits, such as exosomes/secretome.

Activated Biomaterials derived from the cell culture techniques as disclosed herein: The process as disclosed in the present disclosure is a scalable set-up where within the controlled confinement of lab, cells will be used to produce biomaterials consisting of extra cellular matrix and cell modulators such as miRNA and hepatocyte growth factor (HGF). These cell modulators will enable suppression of fibrosis and help transparent, scarless corneal wound healing. The aim is to have a xeno-free process to culture the cell for production of the biomaterial and easy separation and retrieval of the desired components. The cells used in the cell farming is for the production of biomaterials only, and ideally should be re-used for multiple cycles post retrieval and bio-material harvesting.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a process for culturing stem cells to obtain a population of expanded stem cells, said method comprising: (a) obtaining a population of stem cells; and (b) culturing the stem cells of step (a) in either a spheroid-based system or a microcarrier-based system, to obtain a population of expanded stem cells, and a stem cell derived-conditioned medium.

In an embodiment of the present disclosure, there is provided a process for culturing mesenchymal stem cells to obtain a population of expanded mesenchymal stem cells, said method comprising: (a) obtaining a population of mesenchymal stem cells; and (b) culturing the mesenchymal stem cells of step (a) in either a spheroid-based system or a microcarrier-based system, to obtain a population of expanded mesenchymal stem cells, and a mesenchymal stem cell derived-conditioned medium.

In an embodiment of the present disclosure, there is provided a process for culturing stem cells to obtain a population of expanded stem cells, said method comprising: (a) obtaining a population of stem cells; and (b) culturing the stem cells of step (a) in either a spheroid-based system or a microcarrier-based system, to obtain a population of expanded stem cells, and a stem cell derived-conditioned medium, wherein the culturing comprises culturing the stem cells of step (a) in a culture medium comprising a corneal stromal stem cell derived-conditioned medium, and wherein the corneal stromal stem cell derived-conditioned medium is obtained by culturing corneal limbal stem cells.

In an embodiment of the present disclosure, there is provided a process for culturing mesenchymal stem cells to obtain a population of expanded mesenchymal stem cells, said method comprising: (a) obtaining a population of mesenchymal stem cells; and (b) culturing the mesenchymal stem cells of step (a) in either a spheroid-based system or a microcarrier-based system, to obtain a population of expanded mesenchymal stem cells, and a mesenchymal stem cell derived-conditioned medium, wherein the culturing comprises culturing the mesenchymal stem cells of step (a) in a culture medium comprising a corneal stromal stem cell derived-conditioned medium, and wherein the corneal stromal stem cell derived-conditioned medium is obtained by culturing corneal limbal stem cells.

In an embodiment of the present disclosure, there is provided a process for obtaining a mesenchymal stem cell derived-conditioned medium, said process comprising: (a) obtaining a population of mesenchymal stem cells; and (b) culturing the mesenchymal stem cells of step (a) in either a spheroid-based system or a microcarrier-based system, to obtain a population of expanded mesenchymal stem cells, and a mesenchymal stem cell derived-conditioned medium.

In an embodiment of the present disclosure, there is provided a process for culturing stem cells, to obtain a population of expanded stem cells, said process comprising: (a) obtaining a population of stem cells; (b) obtaining microcarriers comprising crosslinked alginate core and crosslinked gelatin surface; (c) suspending the microcarriers in a culture medium, to obtain a suspension; (d) seeding the suspension with the population of stem cells of step (a); (e) culturing the stem cells of step (d) in a culture medium to obtain a population of expanded stem cells adhered to the microcarriers, and a stem cell derived-conditioned medium; and (f) dissolving the microcarriers of step (e) by contacting the microcarriers with a dissolution buffer comprising sodium chloride and trisodium citrate, to obtain a population of expanded stem cell.

In an embodiment of the present disclosure, there is provided a process for culturing mesenchymal stem cells, to obtain a population of expanded mesenchymal stem cells, said process comprising: (a) obtaining a population of mesenchymal stem cells; (b) obtaining microcarriers comprising crosslinked alginate core and crosslinked gelatin surface; (c) suspending the microcarriers in a culture medium, to obtain a suspension; (d) seeding the suspension with the population of mesenchymal stem cells of step (a); (e) culturing the mesenchymal stem cells of step (d) in a culture medium to obtain a population of expanded mesenchymal stem cells adhered to the microcarriers, and a mesenchymal stem cell derived-conditioned medium; and (f) dissolving the microcarriers of step (e) by contacting the microcarriers with a dissolution buffer comprising sodium chloride and trisodium citrate, to obtain a population of expanded mesenchymal stem cell.

In an embodiment of the present disclosure, there is provided a process for culturing stem cells, to obtain a population of expanded stem cells as described herein, wherein the microcarriers are in a size ranging from 50-500 μm. In another embodiment, the microcarriers are in a size ranging from 100-450 μm, or 150-400 μm, or 175-500 μm, or 200-500 μm, or 100-250 μm, or 340-480 μm.

In an embodiment of the present disclosure, there is provided a process for culturing mesenchymal stem cells, to obtain a population of expanded mesenchymal stem cells as described herein, wherein the microcarriers are in a size ranging from 50-500 μm. In another embodiment, the microcarriers are in a size ranging from 100-450 μm, or 150-400 μm, or 175-500 μm, or 200-500 μm, or 100-250 μm, or 340-480 μm.

In an embodiment of the present disclosure, there is provided a process for culturing stem cells, to obtain a population of expanded stem cells as described herein, wherein the microcarriers comprise sodium alginate in the concentration range of 0.01-20% w/v, and gelatin in the concentration range of 0.1-20% w/v. In another embodiment, the microcarriers comprise sodium alginate in the concentration range of 0.05-15% w/v, or 0.075-12.5% w/v, or 1-15% w/v, or 1-12% w/v, or 1-10% w/v, or 0.075-8% w/v, or 1-5% w/v, or 1-3% w/v, and gelatin in the concentration range of 0.05-15% w/v, or 0.075-12.5% w/v, or 1-15% w/v, or 1-12% w/v, or 1-10% w/v, or 0.075-8% w/v, or 1-5% w/v, or 1-3% w/v.

In an embodiment of the present disclosure, there is provided a process for culturing mesenchymal stem cells, to obtain a population of expanded mesenchymal stem cells as described herein, wherein the microcarriers comprise sodium alginate in the concentration range of 0.01-20% w/v, and gelatin in the concentration range of 0.1-20% w/v. In another embodiment, the microcarriers comprise sodium alginate in the concentration range of 0.05-15% w/v, or 0.075-12.5% w/v, or 1-15% w/v, or 1-12% w/v, or 1-10% w/v, or 0.075-8% w/v, or 1-5% w/v, or 1-3% w/v, and gelatin in the concentration range of 0.05-15% w/v, or 0.075-12.5% w/v, or 1-15% w/v, or 1-12% w/v, or 1-10% w/v, or 0.075-8% w/v, or 1-5% w/v, or 1-3% w/v.

In an embodiment of the present disclosure, there is provided a process for culturing stem cells, to obtain a population of expanded stem cells as described herein, wherein the microcarriers are obtained by a method as described in the present disclosure, and wherein the method uses di- or trivalent ions selected from the group consisting of Ca²⁺, Ba²⁺, Fe²⁺, Cu²⁺, Sr²⁺, Fe³⁺, and combinations thereof in a concentration range of 0.01-1000 mM, EDTA in a concentration range of 0.1-100 mM, glutaraldehyde in a concentration range of 0.01-10% v/v, glycine in a concentration range of 1-1000 mg/ml with a crosslinking time of 10 seconds to 60 minutes.

In an embodiment of the present disclosure, there is provided a process for culturing mesenchymal stem cells, to obtain a population of expanded mesenchymal stem cells as described herein, wherein the microcarriers are obtained by a method as described in the present disclosure, and wherein the method uses di- or trivalent ions selected from the group consisting of Ca²⁺, Ba²⁺, Fe²⁺, Cu²⁺, Sr²⁺, Fe³⁺, and combinations thereof in a concentration range of 0.01-1000 mM, EDTA in a concentration range of 0.1-100 mM, glutaraldehyde in a concentration range of 0.01-10% v/v, glycine in a concentration range of 1-1000 mg/ml with a crosslinking time of 10 seconds to 60 minutes.

In an embodiment of the present disclosure, there is provided a process for culturing stem cells, to obtain a population of expanded stem cells as described herein, wherein the process is for obtaining a stem cell derived-conditioned medium.

In an embodiment of the present disclosure, there is provided a process for culturing mesenchymal stem cells, to obtain a population of expanded mesenchymal stem cells as described herein, wherein the process is for obtaining a mesenchymal stem cell derived-conditioned medium.

In an embodiment of the present disclosure, there is provided a process for culturing stem cells, to obtain a population of expanded stem cells, said process comprising: (a) obtaining a population of stem cells; (b) obtaining microcarriers comprising crosslinked alginate core and crosslinked gelatin surface; (c) suspending the microcarriers in a culture medium, to obtain a suspension; (d) seeding the suspension with the population of stem cells of step (a); (e) culturing the stem cells of step (d) in a culture medium to obtain a population of expanded stem cells adhered to the microcarriers, and a stem cell derived-conditioned medium; and (f) dissolving the microcarriers of step (e) by contacting the microcarriers with a dissolution buffer comprising sodium chloride and trisodium citrate, to obtain a population of expanded stem cell, wherein culturing the stem cells of step (d) is done in a culture medium comprising a corneal stromal stem cell derived-conditioned medium, and wherein the corneal stromal stem cell derived-conditioned medium is obtained by culturing of corneal stromal stem cells. In another embodiment, culturing the stem cells of step (d) is done in a culture medium comprising 5-50% volume of the corneal stromal stem cell derived-conditioned medium with respect to the culture medium. In yet another embodiment, the corneal stromal stem cell derived-conditioned medium used is in a range of 7-45%, or 10-50%, or 10-40%, or 15-30%, or 17-28%.

In an embodiment of the present disclosure, there is provided a process for culturing mesenchymal stem cells, to obtain a population of expanded mesenchymal stem cells, said process comprising: (a) obtaining a population of mesenchymal stem cells; (b) obtaining microcarriers comprising crosslinked alginate core and crosslinked gelatin surface; (c) suspending the microcarriers in a culture medium, to obtain a suspension; (d) seeding the suspension with the population of mesenchymal stem cells of step (a); (e) culturing the mesenchymal stem cells of step (d) in a culture medium to obtain a population of expanded mesenchymal stem cells adhered to the microcarriers, and a mesenchymal stem cell derived-conditioned medium; and (f) dissolving the microcarriers of step (e) by contacting the microcarriers with a dissolution buffer comprising sodium chloride and trisodium citrate, to obtain a population of expanded mesenchymal stem cell, wherein culturing the mesenchymal stem cells of step (d) is done in a culture medium comprising a corneal stromal stem cell derived-conditioned medium, and wherein the corneal stromal stem cell derived-conditioned medium is obtained by culturing of corneal stromal stem cells. In another embodiment, culturing the mesenchymal stem cells of step (d) is done in a culture medium comprising 5-50% volume of the corneal stromal stem cell derived-conditioned medium with respect to the culture medium. In yet another embodiment, the corneal stromal stem cell derived-conditioned medium used is in a range of 7-45%, or 10-50%, or 10-40%, or 15-30%, or 17-28%.

In an embodiment of the present disclosure, there is provided a process for culturing mesenchymal stem cells, to obtain a population of expanded mesenchymal stem cells, said process comprising: (a) obtaining a population of mesenchymal stem cells; (b) obtaining microcarriers comprising crosslinked alginate core and crosslinked gelatin surface; (c) suspending the microcarriers in a culture medium, to obtain a suspension; (d) seeding the suspension with the population of mesenchymal stem cells of step (a); (e) culturing the mesenchymal stem cells of step (d) in a culture medium to obtain a population of expanded mesenchymal stem cells adhered to the microcarriers, and a mesenchymal stem cell derived-conditioned medium; and (f) dissolving the microcarriers of step (e) by contacting the microcarriers with a dissolution buffer comprising sodium chloride and trisodium citrate, to obtain a population of expanded mesenchymal stem cell, wherein the corneal stromal stem cell derived-conditioned medium is obtained by a process comprising: (i) obtaining a limbal ring tissue from a human donor cornea; (ii) mincing the tissue, to obtain fragments in the size ranging from 1-2 mm; (iii) suspending the fragments in an incomplete medium, to obtain a suspension; (iv) subjecting the fragments to digestion in the presence of at least one type of collagenase enzyme at a concentration range of 5-20 IU/μl with respect to the suspension, to obtain digested explants; (v) culturing the digested explants in a complete medium comprising 1-10% human platelet lysate for a period of 10-14 days, to obtain a population of corneal limbal stem cells; and (vi) passaging the corneal stromal stem cells of step (e) for a period of 10-14 days, to obtain expanded corneal stromal stem cells and a corneal stromal stem cell derived-conditioned medium. In another embodiment, culturing the mesenchymal stem cells of step (d) is done in a culture medium comprising 5-50% volume of the corneal stromal stem cell derived-conditioned medium with respect to the culture medium. In yet another embodiment, the corneal stromal stem cell derived-conditioned medium used is in a range of 7-45%, or 10-50%, or 10-40%, or 15-30%, or 17-28%.

In an embodiment of the present disclosure, there is provided a process for culturing stem cells, to obtain a population of expanded stem cells, said process comprising: (a) obtaining a population of stem cells; (b) pelleting the stem cells of step (a), to obtain a stem cell pellet; (c) resuspending the stem cell pellet in a culture medium comprising basal medium, to obtain a stem cell suspension; (d) obtaining stem cell spheroids from the stem cell suspension obtained in step (c), wherein the stem cell spheroids are having a density of stem cells in a range of 600-10,000 cells per spheroid; and (e) culturing the stem cell spheroids of step (d) in a culture medium comprising basal medium to obtain a population of expanded stem cells, and a stem cell derived-conditioned medium.

In an embodiment of the present disclosure, there is provided a process for culturing mesenchymal stem cells, to obtain a population of expanded mesenchymal stem cells, said process comprising: (a) obtaining a population of mesenchymal stem cells; (b) pelleting the mesenchymal stem cells of step (a), to obtain a mesenchymal stem cell pellet; (c) resuspending the mesenchymal stem cell pellet in a culture medium comprising MSC basal medium, to obtain a mesenchymal stem cell suspension; (d) obtaining mesenchymal stem cell spheroids from the mesenchymal stem cell suspension obtained in step (c), wherein the mesenchymal stem cell spheroids are having a density of mesenchymal stem cells in a range of 600-10,000 cells per spheroid; and (e) culturing the mesenchymal stem cell spheroids of step (d) in a culture medium comprising MSC basal medium to obtain a population of expanded mesenchymal stem cells, and a mesenchymal stem cell derived-conditioned medium.

In an embodiment of the present disclosure, there is provided a process for culturing mesenchymal stem cells, to obtain a population of expanded mesenchymal stem cells, said process comprising: (a) obtaining a population of mesenchymal stem cells; (b) pelleting the mesenchymal stem cells of step (a), to obtain a mesenchymal stem cell pellet; (c) resuspending the mesenchymal stem cell pellet in a culture medium comprising MSC basal medium, to obtain a mesenchymal stem cell suspension; (d) obtaining mesenchymal stem cell spheroids from the mesenchymal stem cell suspension obtained in step (c), wherein the mesenchymal stem cell spheroids are having a density of mesenchymal stem cells in a range of 600-10,000 cells per spheroid; and (e) culturing the mesenchymal stem cell spheroids of step (d) in a culture medium comprising MSC basal medium to obtain a population of expanded mesenchymal stem cells, and a mesenchymal stem cell derived-conditioned medium, wherein the process is a spheroid-based process for culturing the mesenchymal stem cells.

In an embodiment of the present disclosure, there is provided a process for culturing mesenchymal stem cells, to obtain a population of expanded mesenchymal stem cells, said process comprising: (a) obtaining a population of mesenchymal stem cells; (b) pelleting the mesenchymal stem cells of step (a), to obtain a mesenchymal stem cell pellet; (c) resuspending the mesenchymal stem cell pellet in a culture medium comprising MSC basal medium and methyl cellulose in a concentration range of 0.2-2% with respect to the culture medium, to obtain a mesenchymal stem cell suspension; (d) obtaining mesenchymal stem cell spheroids from the mesenchymal stem cell suspension obtained in step (c), wherein the mesenchymal stem cell spheroids are having a density of mesenchymal stem cells in a range of 600-10,000 cells per spheroid; and (e) culturing the mesenchymal stem cell spheroids of step (d) in a culture medium comprising MSC basal medium and methyl cellulose in a concentration range of 0.2-2% with respect to the culture medium to obtain a population of expanded mesenchymal stem cells, and a mesenchymal stem cell derived-conditioned medium.

In an embodiment of the present disclosure, there is provided a process for culturing mesenchymal stem cells, to obtain a population of expanded mesenchymal stem cells, said process comprising: (a) obtaining a population of mesenchymal stem cells; (b) pelleting the mesenchymal stem cells of step (a), to obtain a mesenchymal stem cell pellet; (c) resuspending the mesenchymal stem cell pellet in a culture medium comprising MSC basal medium, to obtain a mesenchymal stem cell suspension; (d) obtaining mesenchymal stem cell spheroids from the mesenchymal stem cell suspension obtained in step (c), wherein the mesenchymal stem cell spheroids are having a density of mesenchymal stem cells in a range of 600-10,000 cells per spheroid; and (e) culturing the mesenchymal stem cell spheroids of step (d) in a culture medium comprising MSC basal medium and methyl cellulose in a concentration range of 0.2-2% with respect to the culture medium to obtain a population of expanded mesenchymal stem cells, and a mesenchymal stem cell derived-conditioned medium.

In an embodiment of the present disclosure, there is provided a process for culturing mesenchymal stem cells, to obtain a population of expanded mesenchymal stem cells, said process comprising: (a) obtaining a population of mesenchymal stem cells; (b) pelleting the mesenchymal stem cells of step (a), to obtain a mesenchymal stem cell pellet; (c) resuspending the mesenchymal stem cell pellet in a culture medium comprising MSC basal medium and methyl cellulose in a concentration range of 0.2-2% with respect to the culture medium, to obtain a mesenchymal stem cell suspension; (d) obtaining mesenchymal stem cell spheroids from the mesenchymal stem cell suspension obtained in step (c), wherein the mesenchymal stem cell spheroids are having a density of mesenchymal stem cells in a range of 600-10,000 cells per spheroid; and (e) culturing the mesenchymal stem cell spheroids of step (d) in a culture medium comprising MSC basal medium to obtain a population of expanded mesenchymal stem cells, and a mesenchymal stem cell derived-conditioned medium.

In an embodiment of the present disclosure, there is provided a process for culturing stem cells, to obtain a population of expanded stem cells as described herein, wherein the process is for obtaining a stem cell derived-conditioned medium.

In an embodiment of the present disclosure, there is provided a process for culturing mesenchymal stem cells, to obtain a population of expanded mesenchymal stem cells as described herein, wherein the process is for obtaining a mesenchymal stem cell derived-conditioned medium.

In an embodiment of the present disclosure, there is provided a process for culturing stem cells, to obtain a population of expanded stem cells as described herein, wherein obtaining stem cell spheroids is either done by a static hanging drop method or by spontaneous aggregation of the stem cells.

In an embodiment of the present disclosure, there is provided a process for culturing mesenchymal stem cells, to obtain a population of expanded mesenchymal stem cells as described herein, wherein obtaining mesenchymal stem cell spheroids is either done by a static hanging drop method or by spontaneous aggregation of the mesenchymal stem cells.

In an embodiment of the present disclosure, there is provided a process for culturing mesenchymal stem cells, to obtain a population of expanded mesenchymal stem cells as described herein, wherein the process is a spheroid-based process, and wherein the culturing of spheroids of step (e) is done in a medium comprising corneal stromal stem cell derived-conditioned medium, and wherein the corneal stromal stem cell derived-conditioned medium is obtained from culturing of corneal stromal stem cells. In another embodiment, culturing the mesenchymal stem cell spheroids of step (e) is done in a culture medium comprising 5-50% volume of the corneal stromal stem cell derived-conditioned medium with respect to the culture medium. In yet another embodiment, the corneal stromal stem cell derived-conditioned medium used is in a range of 7-45%, or 10-50%, or 10-40%, or 15-30%, or 17-28%.

In an embodiment of the present disclosure, there is provided a process for culturing mesenchymal stem cells, to obtain a population of expanded mesenchymal stem cells as described herein, wherein the process is a spheroid-based process, and wherein the corneal stromal stem cell derived-conditioned medium is obtained by a process comprising: (i) obtaining a limbal ring tissue from a human donor cornea; (ii) mincing the tissue, to obtain fragments in the size ranging from 1 to 2 mm; (iii) suspending the fragments in an incomplete medium, to obtain a suspension; (iv) subjecting the fragments to digestion in the presence of at least one type of collagenase enzyme at a concentration range of 5-20 IU/μl with respect to the suspension, to obtain digested explants; (v) culturing the digested explants in a complete medium comprising 1-3% human platelet lysate for a period of 10-14 days, to obtain a population of corneal stromal stem cells; and (vi) passaging the corneal stromal stem cells of step (e) for a period of 10-14 days, to obtain expanded corneal stromal stem cells and a corneal stromal stem cell derived-conditioned medium. In another embodiment, culturing the mesenchymal stem cell spheroids of step (e) is done in a culture medium comprising 5-50% volume of the corneal stromal stem cell derived-conditioned medium with respect to the culture medium. In yet another embodiment, the corneal stromal stem cell derived-conditioned medium used is in a range of 7-45%, or 10-50%, or 10-40%, or 15-30%, or 17-28%.

In an embodiment of the present disclosure, there is provided a process for culturing mesenchymal stem cells, to obtain a population of expanded mesenchymal stem cells as described herein, wherein the population of mesenchymal stem cells is selected from the group consisting of human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, and induced pluripotent stem cells. It can be contemplated that any type of MSC can be used in the process as described in the present disclosure. Further, corneal stromal stem cells (CSSC), and limbal epithelial stem cells (LESC) can also be cultured for expansion by the process as described herein. It further can be contemplated that the CSSC and LESC can be used as a naive population for further expansion and the aspect of priming with CSSC-CM would not be used in case the two cell populations of CSSC and LESC are taken as starting materials.

In an embodiment of the present disclosure, there is provided a mesenchymal stem cell derived-conditioned medium obtained by the process as described herein.

In an embodiment of the present disclosure, there is provided an expanded mesenchymal stem cell population obtained by the process as described herein.

In an embodiment of the present disclosure, there is provided a composition comprising the mesenchymal stem cell derived-conditioned medium as described herein.

In an embodiment of the present disclosure, there is provided a composition comprising the expanded mesenchymal stem cell population as described herein.

In an embodiment of the present disclosure, there is provided an exosome preparation obtained by a process comprising: (a) harvesting the mesenchymal stem cell derived-conditioned medium as described herein, to obtain a secretome; (b) centrifuging the secretome, to obtain a pellet; (c) dissolving the pellet in a low serum xeno free medium, to obtain a crude solution; (d) performing density gradient ultracentrifugation with the crude solution, to obtain a fraction comprising exosomes; and (e) purifying the fraction comprising the exosomes by size exclusion chromatography, to obtain an exosome preparation. In another embodiment, the ultracentrifugation is an iodixanol density gradient ultracentrifugation, and the size exclusion chromatography is done by using Captocore 700 columns. In yet another embodiment, the ultracentrifugation is performed by 30% sucrose cushion, and the size exclusion chromatography is done by using Captocore 700 columns.

In an embodiment of the present disclosure, there is provided an exosome preparation obtained by a process comprising: (a) harvesting the mesenchymal stem cell derived-conditioned medium as described herein, to obtain a secretome; (b) centrifuging the secretome, to obtain a pellet; (c) dissolving the pellet in a low serum xeno free medium, to obtain a crude solution; and (d) performing density gradient ultracentrifugation with the crude solution, to obtain an exosome preparation. In another embodiment, the ultracentrifugation is an iodixanol density gradient ultracentrifugation. In yet another embodiment, the ultracentrifugation is performed by 30% sucrose cushion, and the size exclusion chromatography is done by using Captocore 700 columns. It can be contemplated that the method or purification of exosomes is based on the quality of the end-product exosomes that is required.

In an embodiment of the present disclosure, there is provided a composition comprising at least two components selected from the group consisting of: (a) the expanded mesenchymal stem cell population as described herein, (b) the mesenchymal stem cell derived-conditioned medium as described herein, and (c) the exosome preparation obtained by the process as described herein.

In an embodiment of the present disclosure, there is provided a composition comprising: (i) the expanded mesenchymal stem cell population as described herein, and (ii) the mesenchymal stem cell derived-conditioned medium as described herein.

In an embodiment of the present disclosure, there is provided a composition comprising: (i) the expanded mesenchymal stem cell population as described herein, and (ii) the exosome preparation obtained by the process as described herein.

In an embodiment of the present disclosure, there is provided a composition comprising: (i) the mesenchymal stem cell derived-conditioned medium as described herein, and (ii) the exosome preparation obtained by the process as described herein.

In an embodiment of the present disclosure, there is provided a method for treating a condition selected from the group consisting of corneal disorders, liver fibrosis, and hyper-inflammatory conditions, said method comprising: (a) obtaining the exosomes as described herein; and (b) administering the exosomes to a subject for treating the condition.

In an embodiment of the present disclosure, there is provided a method for treating a condition selected from the group consisting of corneal disorders, liver fibrosis, and hyper-inflammatory conditions, said method comprising: (a) obtaining the mesenchymal stem cell derived-conditioned medium as described herein; and (b) administering a therapeutically effective amount of the conditioned medium to a subject for treating the condition.

In an embodiment of the present disclosure, there is provided a method for treating a condition selected from the group consisting of corneal disorders, liver fibrosis, and hyper-inflammatory conditions, said method comprising: (a) obtaining the expanded mesenchymal stem cell population as described herein; and (b) administering a therapeutically effective amount of the expanded mesenchymal stem cell population to a subject for treating the condition.

In an embodiment of the present disclosure, there is provided a method for treating a condition selected from the group consisting of corneal disorders, liver fibrosis, and hyper-inflammatory conditions, said method comprising: (a) obtaining a composition comprising at least two components selected from the group consisting of: (i) the expanded mesenchymal stem cell population as described herein, (ii) the mesenchymal stem cell derived-conditioned medium as described herein, and (iii) the exosome preparation obtained by the process as described herein; and (b) administering a therapeutically effective amount of the composition to a subject for treating the condition.

In an embodiment of the present disclosure, there is provided a composition comprising at least two components selected from the group consisting of: (a) the expanded mesenchymal stem cell population as described herein, (b) the mesenchymal stem cell derived-conditioned medium as described herein, and (c) the exosome preparation obtained by the process as described herein for use in treating a condition selected from the group consisting of corneal disorders, liver fibrosis, and hyper-inflammatory conditions.

In an embodiment of the present disclosure, there is provided an expanded mesenchymal stem cell population obtained by the process as described herein for use in treating a condition selected from the group consisting of corneal disorders, liver fibrosis, and hyper-inflammatory conditions.

In an embodiment of the present disclosure, there is provided a mesenchymal stem cell derived-conditioned medium obtained by the process as described herein for use in treating a condition selected from the group consisting of corneal disorders, liver fibrosis, and hyper-inflammatory conditions.

In an embodiment of the present disclosure, there is provided an exosome preparation as described herein for use in treating a condition selected from the group consisting of corneal disorders, liver fibrosis, and hyper-inflammatory conditions.

In an embodiment of the present disclosure, there is provided a process for isolating and culturing corneal limbal stem cells, to obtain an expanded corneal stromal stem cell population, said process comprising: (a) obtaining a limbal ring tissue from a human donor cornea; (b) mincing the tissue, to obtain tissue fragments; (c) suspending the fragments in an incomplete medium, to obtain a suspension; (d) subjecting the fragments to digestion in the presence of at least one type of collagenase enzyme at a concentration range of 5-20 IU/μl with respect to the suspension, to obtain digested explants; (e) culturing the digested explants in a complete medium comprising 1-10% human platelet lysate for a period of 10-14 days, to obtain a population of corneal limbal stem cells; and (f) passaging the corneal limbal stem cells of step (e) for a period of 10-14 days, to obtain an expanded corneal stromal stem cell population, and a corneal stromal stem cell derived-conditioned medium. In another embodiment, the process is for obtaining a corneal stromal stem cell derived-conditioned medium. In yet another embodiment, the at least one type of collagenase enzyme is a combination of collagenase-I and collagenase-II, and wherein the incomplete medium comprises Minimum Essential Medium, and wherein the collagenase-I and collagenase-II are present in a ratio range of 0.3:1 to 0.5:1. In an alternate embodiment, the donor cornea is a single cornea, and wherein the corneal limbal stem cells obtained in step (e) is in a range of 0.5-1 million cells, and wherein the corneal limbal stem cells obtained in step (e) is a heterogenous cell population comprising corneal stromal stem cells, and limbal epithelial stem cells. In one another embodiment, the donor cornea is a single cornea, and wherein the expanded corneal stromal stem cell population obtained in step (f) is in a range of 4-6 million cells, and wherein the expanded corneal stem cell population obtained in step (f) is an enriched corneal stromal stem cell population. In one another embodiment, wherein the complete medium further comprises Minimum Essential Medium, insulin, transferrin, selenium, and epidermal growth factor. In yet another alternate embodiment, the tissue fragments have a size in a range of 1-2 mm. In one another embodiment, the process as described herein is xeno-free and scalable.

In an embodiment of the present disclosure, there is provided a corneal stromal stem cell derived-conditioned medium obtained from the process as described herein.

In an embodiment of the present disclosure, there is provided an expanded corneal stromal stem cell population obtained from the process as described herein.

In an embodiment of the present disclosure, there is provided a composition comprising the corneal stromal stem cell derived-conditioned medium as described herein.

In an embodiment of the present disclosure, there is provided a composition comprising the expanded corneal stromal stem cell population as described herein.

In an embodiment of the present disclosure, there is provided an exosome preparation obtained by a process comprising: (a) harvesting the corneal stromal stem cell derived-conditioned medium as described herein, to obtain a secretome; (b) centrifuging the secretome, to obtain a pellet; (c) dissolving the pellet in a low serum xeno free medium, to obtain a crude solution; and (d) performing density gradient ultracentrifugation with the crude solution, to obtain an exosome preparation. In another embodiment, the ultracentrifugation is an iodixanol density gradient ultracentrifugation. In yet another embodiment, the ultracentrifugation is performed by 30% sucrose cushion, and the size exclusion chromatography is done by using Captocore 700 columns. It can be contemplated that the method or purification of exosomes is based on the quality of the end-product exosomes that is required.

In an embodiment of the present disclosure, there is provided a composition comprising at least two components selected from the group consisting of: (a) the expanded stem cell population as described herein, (b) the stem cell derived-conditioned medium as described herein, (c) the exosome preparation as described herein, (d) the expanded corneal stromal stem cell population as described herein, and (e) the corneal stromal stem cell derived-conditioned medium as described herein.

In an embodiment of the present disclosure, there is provided a method for treating a condition selected from the group consisting of corneal disorders, liver fibrosis, and hyper-inflammatory conditions, said method comprising: (i) obtaining the composition comprising at least two components selected from the group consisting of: (a) the expanded stem cell population as described herein, (b) the stem cell derived-conditioned medium as described herein, (c) the exosome preparation as described herein, (d) the expanded corneal stromal stem cell population as described herein, and (e) the corneal stromal stem cell derived-conditioned medium as described herein; and (ii) administering the exosomes to a subject for treating the condition.

In an embodiment of the present disclosure, there is provided a method for treating a condition selected from the group consisting of corneal disorders, liver fibrosis, and hyper-inflammatory conditions, said method comprising: (i) obtaining the expanded corneal stromal stem cell population as described herein; and (ii) administering the corneal stromal stem cell population to a subject for treating the condition.

In an embodiment of the present disclosure, there is provided a method for treating a condition selected from the group consisting of corneal disorders, liver fibrosis, and hyper-inflammatory conditions, said method comprising: (i) obtaining the corneal stromal stem cell derived-conditioned medium as described herein; and (ii) administering the corneal stromal stem cell derived-conditioned medium to a subject for treating the condition.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Source of Stem Cells

For the purpose of the present disclosure, mesenchymal stem cells derived from the sources such as bone marrow (BM), corneal limbal stem cells, umbilical cord (UC), Wharton's jelly (WJ), dental pulp (DP) and adipose tissue (AD), corneal limbal stem cell-derived conditioned media primed MSCs can be used in the methods and cell-derived products as described herein. The choice of the stem cell type would be target indication and tissue specific.

Source of Immortalized Adult Stem Cell Lines (Non-Viral Immortalized MSC Cell Lines):

Telomerized human Bone marrow derived mesenchymal stem cell line (BM-MSC/TERT277) was developed from mesenchymal stem cells isolated from spongy bone (sternum) by non-viral gene transfer of a plasmid carrying the hTERT gene. Positively transfected cells were selected by using neomycin phosphotransferase as selectable marker and Geneticin sulfate addition. The cell line was continuously cultured for more than 25 population doublings without showing signs of growth retardation or replicative senescence.

Telomerized human Wharton's Jelly derived mesenchymal stem cell line (WJ-MSC/TERT273) was established under xeno-free conditions from primary tissue disaggregation to non-viral transfer of hTERT.

The cell lines were characterized by unlimited growth while maintaining expression of cell type specific markers and functions such as: (i) typical mesenchymal morphology; (ii) expression of typical mesenchymal stem cell markers such as CD73, CD90 and CD105; (iii) differentiation potential towards adipocytes, chondrocytes, osteoblasts; and (iv) production of extracellular vesicles with angiogenic and anti-inflammatory activity.

Culture medium used—The culture medium used for culturing the mesenchymal stem cells comprises low serum xenofree medium supplemented with human platelet lysate (0-2%) and combination of 1-2 mM Glutamine, human Epidermal Growth Factor (1-50 ng/ml), Insulin, Transferrin, Selenium, Platelet derived growth Factor (10-100 ng/ml), bFibroblast Growth Factor (1-50 ng/ml), Hydrocortisone (10-100 mM), dexamethasone (0.01-1 mM), Ascorbic acid-2-phosphate (0.01-1 mM), and Insulin Growth Factor (1-50 ng/ml).

Minimum Essential medium—The MEM used for the culturing of CSSC comprises MEM along with low serum xenofree medium supplemented with human platelet lysate (0-2%) and combination of 1-2 mM Glutamine, human Epidermal Growth Factor (1-50 ng/ml), Insulin, Transferrin, Selenium, Platelet derived growth Factor (10-100 ng/ml), bFibroblast Growth Factor (1-50 ng/ml), Hydrocortisone (10-100 mM), dexamethasone (0.01-1 mM), Ascorbic acid-2-phosphate (0.01-1 mM), and Insulin Growth Factor (1-50 ng/ml).

The complete medium specifically for the working example: MEM+2% HPL, 1× Insulin-Transferrin-Selenium (ITS), 10 ng/ml Epidermal growth factor (EGF)+Antibiotics (1× Penicillin Stretomycin (Gibco).

The incomplete medium is the complete medium devoid of human platelet lysate (HPL)

The below table describes the different kinds of medium that can be used for culturing of the stem cells as per the present disclosure.

| Cell type | Components |
|---|---|
| BMMSC, ADMSC, | Combination of one or more of: Commercially available |
| DPMSC, UCSMC, WJMSC | media described below + (1-10%) and combination of 1-2 mM |
| | Glutamine, Insulin, Transferrin, Selenium, Platelet derived |
| | growth Factor (10-100 ng/ml), bFibroblast Growth Factor (1- |
| | 50 ng/ml) |
| CSSC, LESC | Combination of one or more of: Commercially available media |
| | described below + (1-10%) and combination of 1-2 mM |
| | Glutamine, human Epidermal Growth Factor (1-50 ng/ml), |
| | Insulin, Transferrin, Selenium, Platelet derived growth Factor |
| | (10-100 ng/ml), bFibroblast Growth Factor (1-50 ng/ml), |
| | Hydrocortisone (10-100 mM), dexamethasone (0.01-1 mM), |
| | Ascorbic acid-2-phosphate (0.01-1 mM), Insulin Growth Factor |
| | (1-50 ng/ml) |
| Commercially available | MEM (Gibco), DMEM (high or low glucose) (Gibco), Eagle's |
| media for all cell types | basal medium, Ham's F10 medium (F10) (Gibco), Ham's F-12 |
| including iPSCs | medium (F12) (Gibco), Iscove's modified Dulbecco's medium |
| | (IMDM) (Gibco), Liebovitz's L-15 medium, MCDB, |
| | DMEM/F12(Gibco), RPMI 1640 (Gibco), advanced DMEM |
| | (Gibco), DMEM/MCDB201 (Sigma), and CELL-GRO FREE, |
| | Mesenchymal Stem Cell Growth Medium (MSCGM), |
| | Mesencult-ACF Plus (StemCell Technologies), EpiLife ™ CF |
| | Kit (Gibco) |

### Example 1

### Isolation and Culture of Corneal Limbal Stem Cells to Obtain an Expanded Population of Corneal Stromal Stem Cells

The present example describes the process for isolating, culturing and expanding the corneal limbal stem cells under the xenofree culture conditions to obtain an enriched and expanded population of CSSC. The corneal limbal stem cells are isolated from the limbal region of the cornea. The two major sub-populations of corneal limbal stem cells are CSSC and limbal epithelial stem cells (LESC). The process as disclosed in the present disclosure specifically enriches the heterogenous population of CSSC and LESC obtained in passage 1 to obtain an enriched and expanded population of CSSC.

CSSCs are type of MSCs derived from the tissues of cornea. The xenofree process for isolation and culture of CSSCs from human donor derived corneas was finalised by testing four variations of xenofree culture protocols, where four different combinations of enzymes for digestion and media for culture were deployed. The main aim was to select the combination of enzyme for digestion and media for culture that would result in obtaining the high-quality yield of CSSCs and high yield of exosomes. For this purpose, following combinations of collagenase enzyme and incomplete media were tested to evaluate the effectiveness of each combination for the isolation of CSSCs from human donor cornea (FIG. 29):

(A): Combination I (LIB_MEM): Digestion with Liberase (LIB)+Minimum Essential Medium (MEM) media (Centre of Cellular Therapy (cGMP) validated).
(B): Combination II (LIB_RB): Digestion with Liberase (LIB)+RoosterBio Xenofree Basal media (RB)
(C) Combination III (COL_RB): Digestion with Collagenase Type IV (COL)+RoosterBio Xenofree Basal media (RB)
(D) Combination IV (COL_MEM): Digestion with Collagenase Type IV (COL)+MEM media (Centre of Cellular Therapy (cGMP) validated) (MEM).

Liberase as used herein a type of collagenase enzyme, which is a combination of collagenase-I and collagenase-II.

To obtain the expanded corneal stromal stem cell population, the present disclosure describes a process for isolating and culturing corneal stem cells using a combination of liberase (collagenase enzymatic digestion) and MEM enzyme under xenofree conditions. The steps of the process are provided below:

(a) Human donor derived corneas were washed with antibiotic fortified buffered saline before extracting limbus which contain the CSSC.
(b) Under aseptic conditions, a 360° limbal ring tissue was excised from the human donor cornea using surgical instruments.
(c) The excised limbal ring tissue was then washed with buffered saline and minced into smaller fragments.
(d) The minced tissue fragments were suspended into incomplete media (MEM or DMEM media) to obtain a suspension.
(e) The minced tissue fragments were subjected to collagenase digestion by adding 20 μL of reconstituted collagenase IV (17104019, Thermofisher) or Liberase (Roche) at a concentration of 5-20 IU/μL with respect to the tissue suspension, to obtain digested explants.
(f) After 16 h of incubation, collagenase enzymatic digestion was stopped by adding 2 mL of complete media fortified with 2% human platelet lysate (HPL).
(g) The digested explants were then spun down at 1000 rpm for 3 min at room temperature, in saline added with penicillin and streptomycin.
(h) At passage 0, the digested explants were resuspended in 5 mL xenofree complete media (MEM+2% HPL, 1× Insulin-Transferrin-Selenium (ITS), 10 ng/ml Epidermal growth factor (EGF)), and antibiotics (1× penicillin (Gibco)) and were cultured in Corning CellBIND flasks for 14 days to obtain the population of high quality corneal stromal stem cells for 14 days. The complete media was changed every 3 days.
(i) At the end of 14 days of passage 1 (P1), the cells isolated from the digested explants were trypsinized with Tryple (1×, Gibco) and resuspended in fresh complete media. The cells were seeded at 10,000 cells/cm² in CellBIND flasks for passages 1 through passage 2 (P2). The cells were then sub-cultured every 5-7 days each.

The expanded high quality CSSCs obtained at P1 and P2 were then characterized using the following markers: (i) Limbal epithelial stem cells (LESC) positive markers: p63a, ABCB5; (ii) Corneal stromal stem cells (CSSC) positive markers: CD90, CD73, CD105, ABCG2; and (iii) CSSC negative markers: a-SMA, CD34, ABCB5, p63-alpha.

For further characterization of CSSC, p63-alpha and ABCB5 (which are Limbal epithelial stem cell (LESC) population markers) were used for demonstrating the purity of the CSSC population isolated by the LIB_MEM process and the enrichment of CSSCs over LESCs from Passage 1 to Passage 2.

### Results

As described above, four combinations of collagenase enzyme and media were tested to evaluate the effectiveness of each combination to obtain high-quality yield of CSSCs from human donor cornea. The CSSCs obtained from each process deploying different combinations of collagenase enzyme and media were characterized based on the expression of CSSC-specific markers (CD90/CD73/CD105).

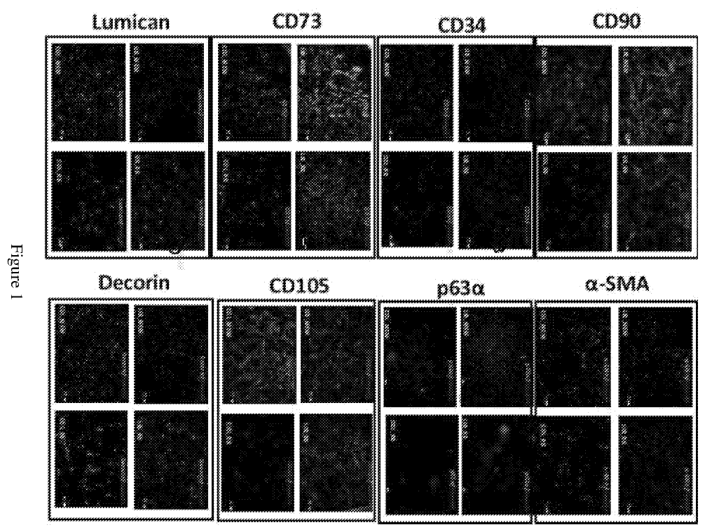

FIG. 1 shows the comparison between the four xenofree process using different combinations to obtain a high-quality yield of CSSCs, wherein the comparison was made in term of the expression of CSSC-specific markers in the CSSC population from each process. Referring to FIG. 1, the CSSCs consistently stained strongly positive for markers including CD90, CD73, CD105 and negative for alpha-SMA, CD34, decorin and lumican for CSSCs isolated by the process using the combination of LIB_MEM (combination 1). The other three processes (i.e., with combination II, III, IV) showed inconsistency in expression across cells and showed relatively lower expression of the positive markers (CD90, CD73, CD105). Therefore, it can be inferred that the process using the combination of LIB_MEM (combination 1) was found to be most suitable for the maintenance of sternness markers in CSSCs, as compared to processes using the combination II, III, and IV respectively.

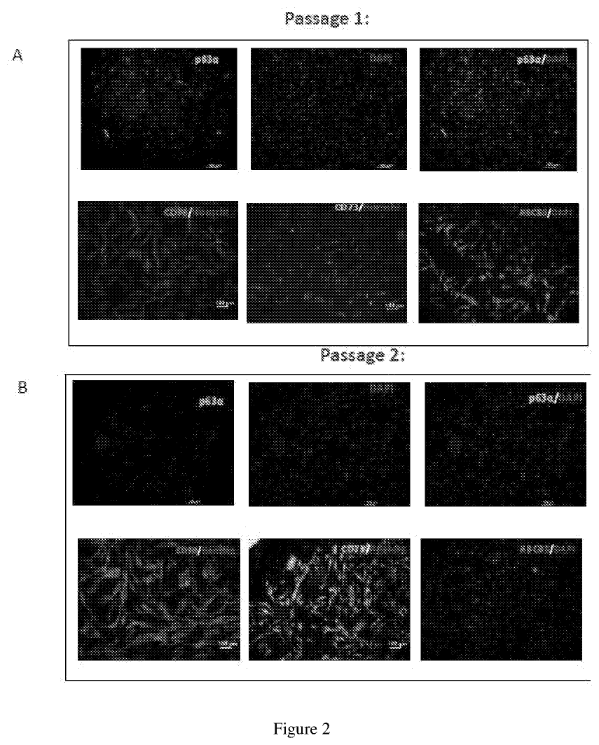

The CSSCs isolated and cultured by the process using the combination of LIB_MEM (combination I) were further characterized, as shown in FIG. 2. The process using the combination II yielded a mix of p63a/ABCB5 positive and negative cells at Passage 1 (FIG. 2A), indicating a mixed population of LESCs (positive stained) and CSSCs (negative stained). CD90 and CD73 were expressed by the stem cells in both passages. The number of CSSC obtained at passage 1 was in the range of 0.5-1 million.

However, at the passage 2 (FIG. 2B), the loss of expression of p63a-alpha and ABCB5, and high expression of CD90 and CD73 in CSSCs indicated the enrichment of CSSCs over LESCs. The enrichment of CSSCs over LESCs resulted in a pure stromal stem cell population. The yield of pure stromal stem cell population obtained at passage 3 was in the range of 4-6 million.

The liberase enzyme as used herein is a combination of collagenase-I and collagenase-II in a ratio range of 0.3:1 to 0.5:1 along with a neutral protease content in a range of 1.8-2.6 mg. The collagenase-I content is in a range of 2.2-3.4 mg and the collagenase-II content is in a range of 1.5-2.3 mg which can be used.

Therefore, it can be inferred from FIG. 1 and FIG. 2 that the isolation and culture of CSSC using the combination I (LIB_MEM) resulted in high-quality yield of CSSCs. The high-quality yield of CSSC can then be further used for clinical applications from Passage 2-3. For clarity, as per the method disclosed herein, the population of corneal limbal stem cells are obtained from the donor cornea. The population of corneal limbal stem cells comprises sub-populations of corneal stromal stem cells (CSSC) and limbal epithelial stem cells (LESC). After the first passage, a heterogenous stem cell population comprising CSSC and LESC was obtained. After a second passage, the CSSC population was selectively enriched to obtain a population of expanded CSSC. Therefore, it can be contemplated that the expanded CSSC population can have a very less percentage of non-CSSC also. Hence, such an expanded CSSC population comprising a small percentage of non-CSSC is also construed to be a part of the present disclosure.

### Example 2

### Culture and Expansion of Mesenchymal Stem Cells

### Culture and Expansion of Primary Human Bone Marrow-Mesenchymal Stem Cells

The present example describes the process for culturing and expansion of hBM-MSC (RoosterBio Inc.) obtained from three donors (Donor ID #D200, D227 and D257). The expanded population of hBM-MSCs were further used for secretome and exosome production. The steps of the process for culturing and expansion of hBM-MSC was carried out by the following:

The hBM-MSC High Performance Media Kit XF was kept at room temperature.

The booster vial and media bottle well were sprayed with 70% isopropyl alcohol before transferring them into biosafety cabinet. The wet surface was wiped with a clean tissue paper.

1 vial (10 ml) hMSC Media Booster XFM (SU-016) was added to 500 ml hMSC High Performance Basal Media (SU-005) by using a serological pipette. Both the media was mixed with the pipettor. About 5-8 ml of complete media was added in to booster vial and was then gently mixed to retain any residual components of the booster.

RoosterVial-hBM-1M-XF was obtained from liquid nitrogen (LN) and was immediately thawed in 37° C. water bath with gentle swirling. The process was monitored. RoosterVial-hBM-1M-XF was then removed from water bath after 2-3 min once the ice was melted.

The vial was sprayed well with 70% isopropyl alcohol before transferring it into the biosafety cabinet. The cells were then aseptically transferred into a 50 mL centrifuge tube.

4 mL of culture media was slowly added dropwise to the cells in the centrifuge tube.

The centrifuge tube was then centrifuged at 200×g for 10 min at room temperature.

The supernatant was carefully removed without disturbing the cell pellet. The cells were then resuspended in 5 mL of complete media.

As a quality control (QC), the cell number was counted and recorded.

After resuspended the cells, the volume was made up to 30 mL with culture media.

The media was mixed properly with the cells, and subsequently the cells were equally seeded into flasks, and more media was added to bring the volume up to the final volume to ensure that the fully coverage of the flask with the media.

The flask was then transferred into a 5% CO₂, 37° C. sterilized incubator.

The culture was microscopically observed every day from day 3 onwards to determine percentage confluency. If culture was found to be less than 50% confluent on day 3, then it led to the change in the media. The spent media was completely removed from the vessel and was replaced with the same volume of the fresh complete media. The vessel was transferred back into the incubator. When culture was found to be >80% confluent, harvesting of the cells was done on the following day.

The media was changed on day 3 followed by every 48 h.

For harvesting, the vessel was transferred into the biosafety cabinet and the spent media was removed. About 10 mL of spent media was collected in sterile container if it was used to quench harvest enzyme.

The media was then removed, and the cells were washed with 1×PBS followed by addition of 10 mL of TrypLE and incubation in 37° C. incubator. The culture was checked every 5 min until the detachment of cells from the surface.

Equal amount of quench (fresh media) or spent media was added to stop the TrypLE activity.

The suspension was then transferred into a sterile 50 ml centrifuge tube. Subsequently, the centrifuge tube was centrifuged at 200×g for 10 min.

The supernatant was aspirated, and the cells were resuspended with 4-5 mL of fresh media. The total volume of cell suspension was then measured.

The well was mixed properly, and 0.1 mL of cells were transferred into microcentrifuge tubes for cell counts. The cells were diluted to 0.5 mL with DPBS to achieve the count of the cells in the range of 0.1-1×10⁶ cells/mL. The well was mixed, and cells were ready for counting with cell counting device.

Using this procedure, the cells can be expanded to 200 million (first passage) and up to 2 billion (second passage).

### Culture and Expansion of Adipose-Derived Mesenchymal Stem Cells (AD-MSC)

The immortalized/telomerised ADMSCs (Cat #ASC/TERT1) were procured from Evercyte and cultured and expanded according to the process described in Example 2, however, Evercyte proprietary xenofree media was used instead of Rooster Bio media. The expanded ADMSCs were characterized using the cell markers CD90, CD73 and ABCG2, and alpha-SMA.

### Culture & Expansion of Umbilical Cord Derived Mesenchymal Stromal Cells (UC-MSC)

The present example describes the process for culturing and expansion of umbilical cord-derived mesenchymal stromal cells.

In this process, fresh Umbilical cords (UCs) were obtained from informed, healthy mothers in local maternity hospitals after normal deliveries and processed immediately. The cords were then rinsed twice in phosphate buffered saline in penicillin and streptomycin, and the cord blood was removed during the process. The washed cords were cut into 1-2 mm pieces and floated in low-glucose Dulbecco's modified Eagle's medium containing 10% fetal bovine serum. The pieces of cord were incubated at 37° C. in a humidified atmosphere consisting of 5% CO2. Nonadherent cells were removed by washing. The medium was replaced every 3 days after the initial plating. When well-developed colonies of fibroblast-like cells appeared after 10 days, the cultures were trypsinized and passaged into a new flask for further expansion. UCMSCs from passage 2-5 were used for clinical applications.

### Results

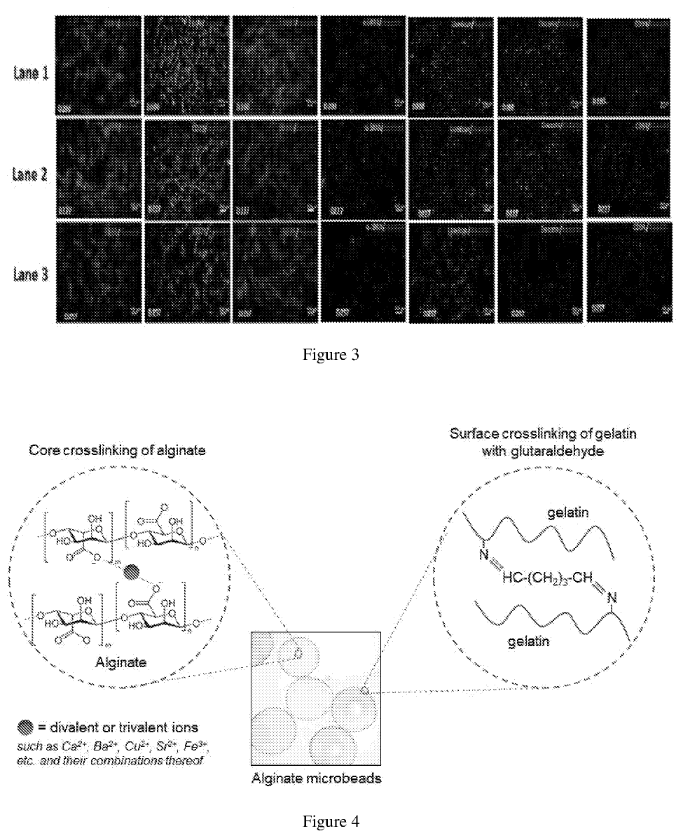

Human BM-MSCs (RoosterBio Inc.) from three donors (Donor ID #D200, D227 and D257) were cultured and expanded for secretome and exosome production, according to the process described above. The Human BM-MSCs were characterized prior to exosome induction to confirm the stemness and integrity of the cells (quality check step). FIG. 3 shows the characterization of Human BM-MSCs. Referring to FIG. 3, it can be observed that all three Human BM-MSCs stained positive for MSC markers including CD90, CD73, CD105 and negative for alpha-SMA, CD34. The Human BM-MSCs expressed low levels of lumican and decorin (extracellular matrix proteins).

Therefore, it can be inferred from FIG. 3 that a high-quality yield of Human BM-MSCs with positive expression of CD90, CD73, CD105 and negative expression of alpha-SMA, CD34. The expanded Human BM-MSCs was further used for the production of high yield of secretomes and exosomes for various clinical applications.

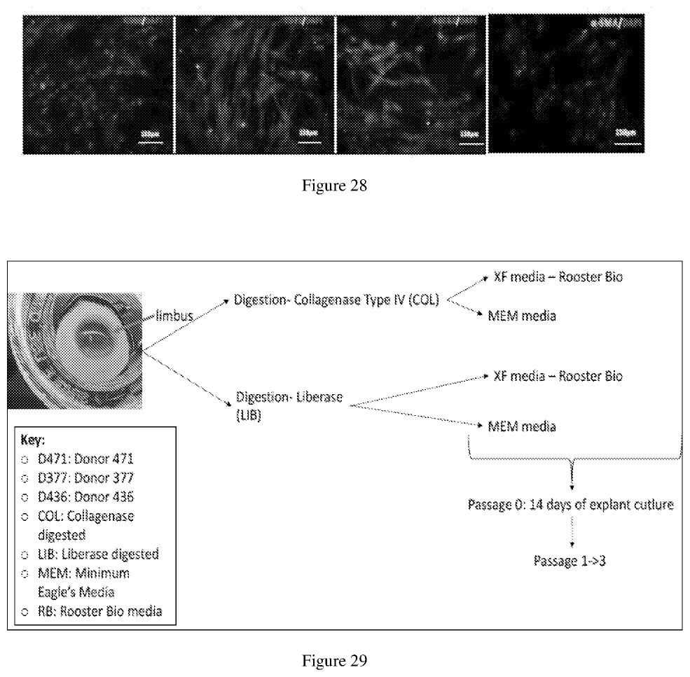

FIG. 28 shows the characterization of immortalized ADMSCs. Referring to FIG. 28, stemness markers, such as, CD90, CD73 and ABCG2 were expressed by the ADMSCs while stress marker alpha-SMA was not expressed by ADMSCs. The positive expression of markers such as CD90, CD73 and ABCG2 and negtative expression of alpha-SMA indicates the isolation and expansion of high-quality yield of ADMSCs population. The expanded ADMSCs were further used for the production of high yield of secretomes and exosomes. These ADMSCs and ADMSC-derived secretomes and exosomes can be then used individually and in combination thereof, as a final product for various clinical applications.

### Example 3

### Expanding Stem Cells in Three-Dimensional (3D) Microcarrier-Based Culture

### Fabrication of Alginate-Gelatin Microcarriers

FIG. 4 depicts the basic concept behind the preparation of Alg/Gel microbeads for 3D culture of cells. Briefly, sodium alginate beads are fabricated by using commonly employed di- or trivalent ions as crosslinking agents, such as Ca²⁺, Ba²⁺, Fe²⁺, Cu²⁺, Sr²⁺, Fe³⁺, or their combinations thereof, to yield solid transparent microspheres. Subsequently, the microbeads ware coated with gelatin which will be reversibly crosslinked with glutaraldehyde. The gelatin coated bead surface facilitates cell adhesion and proliferation as bare alginate beads do no possess cell binding motifs conducive for cell adhesion and growth. Table 1 depicts the different components along with their percentages for obtaining the microcarriers/microbeads.

TABLE 1

| S. No. | Components/Parameters | Working ranges | |
|---|---|---|---|
| 1. | Sodium alginate (low/medium/high | 0.01-20% | w/v |
| | viscosity) | | |
| 2. | Di- or trivalent ions (Ca²⁺, | 0.01-1000 | mM |
| | Ba²⁺, Fe²⁺, Cu²⁺, Sr²⁺, Fe³⁺, | | |
| | and their combinations thereof) | | |
| 3. | EDTA | 0.1-100 | mM |
| 4. | Gelatin (50-400) bloom | 0.1-20% | w/v |
| 5. | Glutaraldehyde | 0.01-10% | v/v |
| 6. | Glycine | 1-1000 | mg/mL |
| 7. | Crosslinking time | 10 s-60 min | |
| 8. | Bead size (diameter) | 50-500 | μm |

As per one of the embodiments, the microcarriers that were synthesised for the present disclosure is as per the below mentioned protocol. Microcarriers—Alginate beads crosslinked with Ca²⁺ and Ba²⁺ ions and gelatin crosslinked with glutaraldehyde

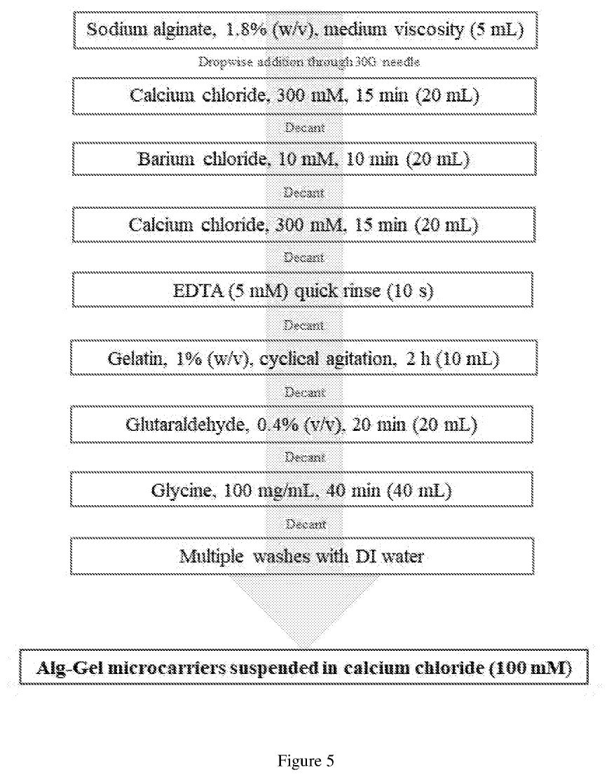

FIG. 5 depicts a flowchart for obtaining the alginate-gelatin based microcarriers used in the present disclosure. As one of the example, the alginate-gelatin based microcarrier system was developed using medium viscosity alginate. Briefly, alginate solution (1.8% w/v) was extruded from a 30G needle into a bath containing calcium chloride solution (300 mM) to crosslink alginate. The crosslinking occurs due to the ionic interaction between the carboxyl groups of two adjacent alginate chains and the calcium ions. This results in the formation of a stable three-dimensional network. The beads so formed were incubated in calcium chloride for 10 min after which the solution was decanted. Subsequently, this step was followed by the suspension of the crosslinked alginate into barium chloride (10 mM) for 10 mins. In order to ensure removal of excess calcium ions from bead surface, the beads were quickly rinsed in EDTA (0.05%) before coating with gelatin (1% w/v). The beads were suspended in gelatin for a period of 2 h with alternate cycles of static (10 mins) and dynamic (2 mins). To facilitate efficient reversible crosslinking of the collagen derivative, glutaraldehyde (0.4% v/v) was used and the beads were incubated in it for 20 mins. Glutaraldehyde reacts with the non-protonated ε-amino groups (—NH2) of lysine or hydroxylysine through a nucleophilic addition-type reaction to yield a crosslinked gelatin coated surface. The beads were then suspended in glycine (100 mg/mL) for 40 mins to remove unreacted glutaraldehyde. In the final step, the beads were washed and suspended in calcium chloride solution (100 mM) for a period of 12 h and stored at 4° C.

The microcarriers obtained by the protocol as described herein, and the cell adhered microcarriers as described herein was evaluated by the parameters mentioned below.

### 1. Circularity Index (CI)

CI was calculated using Image J software (version 2.0.0). Briefly, oval/elliptical tool was used to fit the diameter of the beads and from the measure tool various parameters like perimeter and CI were obtained. From the perimeter value and using the formula 2πr, radius and diameter values were derived.

### 2. Cell Adherence on the Microbeads

To demonstrate cell adherence onto the fabricated Alg/Gel microbeads, 0.5×106 BM-MSCs were statically loaded onto the microbeads (50 mg) in a 24 well plate and were incubated for a period of 24 h. After the incubation period, the beads were observed under a phase contrast microscope.

### 3. Cell Seeding Protocol for Dynamic Culture

Briefly, about 30 mg of each bead type was taken and equilibrated with the media for 30 min in a spinner flask. Subsequently, each bead type was subjected to an alternate cycle of static and dynamic conditions for the first 3 h. The dynamic condition was set for 5 min (done manually for RCP and PS beads) while the static was set for 55 min and this cycle was repeated three times. Then, the microbeads were transferred to spinner flasks and maintained at a constant dynamic condition with stirring speed set to 85 rpm for 24 h. The RCP and the polystyrene beads were pooled in a single spinner flask while the sodium alginate beads were cultured separately in another spinner flask under dynamic condition. After 24 h, the beads were analysed for cell adherence and cell viability.

### 4. Live Dead Assay

Fluorescence based Live/Dead assay based on calcein-AM (Cat. No.: C1430, ThermoFisher) and ethidium homodimer (Cat. No.: 46043, Sigma-Aldrich) was used according to the manufacturers' protocol and imaged using a Laser scanning Confocal Microscope (Nikon C2 with Nis Elements 5.0 Imaging Software). Hoechst (Cat. No: 14533, Sigma Aldrich) staining was used to label nucleus. The live cells were labelled in green, dead cells in red and nuclei in blue. Maximum intensity projections of the Z stacks (spanning about 50 μm) were made using Image J software (version 2.0.0).

5. Cell Viability Testing with Trypan Blue

Cell suspension was diluted in trypan blue (Cat. No.: T8154, Sigma Aldrich) in the ratio of 1:1, and the non-viable cells (in blue) and viable cells (unstained) were counted in a Neubauer chamber to determine the cell viability index.

### 6. Immunostaining

Immunofluorescence staining stem cell markers was done using routine antibody staining protocol. Briefly, adhered cells on the beads were fixed in 10% neutral buffered formalin for 30 mins at room temperature (RT) and washed with PBS containing triton (0.1%) for 5 mins. For blocking, 1% bovine serum albumin (BSA) was used and the samples were incubated for 45 mins at RT. Primary antibody diluted in the blocking buffer was incubated overnight at 4° C. and washed with PBS (3×; 10 minutes each). Secondary antibody diluted in the blocking buffer was incubated for 1 h and washed with PBS (3×; 10 minutes each) and finally incubated with Dapi for 10 min in PBS. Samples were imaged either using a Laser scanning microscope (Nokia C2) or Keyence microscope. Maximum intensity projections of the Z stacks (spanning about 50 um) were made using Image J software (version 2.0.0), wherever applicable.

### Decellularization Protocol & Dissolution of Alg/Gel Microbeads

Cell-laden Alg/Gel microbeads were incubated in a dissolution buffer, which is a combination of sodium chloride (0.15 M) and trisodium citrate (0.055 M) trisodium citrate, over a period of 9 minutes at room temperature. After microbead dissolution, the suspension was centrifuged and the cells were pelleted out. The cells were resuspended in PBS and a trypan blue staining assay was performed to count the number of viable cells.

### Estimated Number of Beads for Bioreactor

As the average radius of Alg/Gel beads is ˜200 μm, the following calculations will be helpful to arrive at the requirements to culture 10 million cells in a volume of 500 mL bioreactor that maintains constant stirring and dynamic culture conditions.

i. Micro sphere/bead radius will be ˜200 μm (diameter=˜400 μm)

According to sphere volume equation=(4/3 π r3), micro sphere volume equal to (3.35×107) (μm)

ii. Therefore, in 1 ml of alginate solution, the numbers of microbeads are calculated to be
a. 1 ml of solution volume equal to 1cc=10₁₂ (μm)₃.
b. 1 ml solution contains=10₁₂ (μm)₃/vol. of each microbead=10₁₂ (μm)₃/(3.35×10₇) (μm)₃=29850=˜3×10₄

Hence number of beads required for the 500 mL bioreactor=3×10₄×500=1.5×10₇.

### Preparation of Microcarriers for Bioreactor

Approximately, 200 g of the microcarriers/beads was weighed in 120 mL of PBS buffer and rehydrate.

The mixture was allowed to hydrate for at least 1 h before heat sterilization by autoclave (121° C. for 15 min).

After heat sterilization, the microcarriers/beads will settle to the bottom and was washed with 50 mL of culture medium. The washing step was repeated twice

After this procedure, microcarriers are ready to use in cell culture.

### Culturing on the Surface of Microcarriers in a 500 ml Bioreactor

The mesenchymal stem cells (MSCs) were grown in sufficient numbers in a two-dimensional (2D) xeno-free culture conditions, and then trypsinised to get a single cell suspension.

A day prior to the experiment, 500 ml spinner flasks or bioreactors was autoclaved if required. If sterile spinner flasks/bioreactors are available, they will be readily used.

The autoclaved/sterile spinner flasks were washed once with 50 mL DPBS. After that, 200 g of microcarriers suspended in 150 mL of xenofree MSCs medium was added to each of the 500 ml spinner flask or bioreactor.

Spinner Flasks or bioreactors were equilibrated for 30 min in a standard tissue culture incubator.

Following that, 10 million MSCs suspended in 50 mL volume were added to each 500 mL flask or bioreactor.

To achieve uniform cell seeding, the spinner flasks or bioreactors were placed on magnetic stirrer plate and initial stirring for 5 min will be started at 10-30 rpm for vertical impellers while 30-8 rpm for horizontal impellers, followed by rest for 55 min, at 37° C. and 5% CO₂, for a total of 1-hour static/dynamic incubation cycle. These cycles will be repeated for four times.

At the end of the seeding, 150 mL of medium was added to the culture and continuous stirring at 15-30 rpm for vertical impellers while 30-85 rpm for horizontal impellers was done.

The total volume will become 400 ml of media with beads and cells.

Half of the total medium volume was changed every day. For this, the beads were allowed to settle to the bottom of the bioreactor and carefully, 200 ml of the medium was carefully aspirated and replaced with fresh xenofree MSCs medium.

The culture was maintained up to 7-14 days.

### Results

### Size Distribution of the Microcarriers

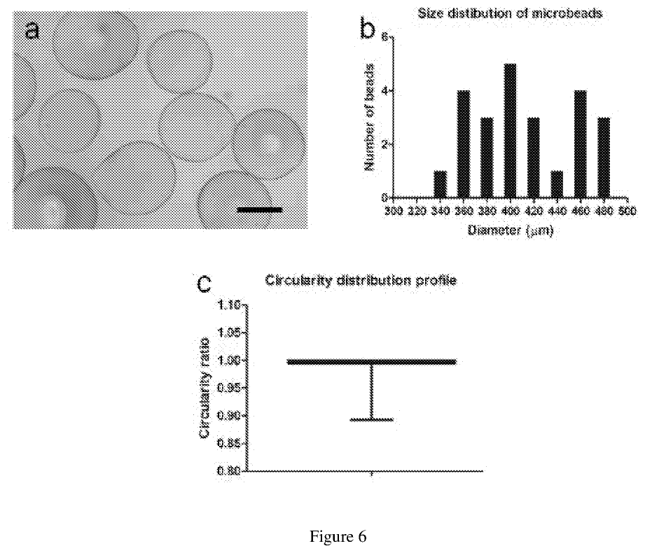

The alginate-gelatin microcarriers were obtained as mentioned previously in Example 3. The size of the microbeads was analyzed using the phase contrast mode of the EVOS imaging system. A batch of microbeads was assessed, and the size distribution of the alginate gelatin beads were plotted using the GraphPad Prism 5 software. In addition, the circularity profile of the microbeads was also analysed (FIG. 6). The size of the microbeads was found to be in the range of 409.84±44.14 μm while the circularity ratio of >0.90 clearly indicates that the shape of the microbeads are more or less a proper sphere (circularity ratio of 1 indicates a perfect sphere).

### Cell Adherence on the Microcarriers/Microbeads

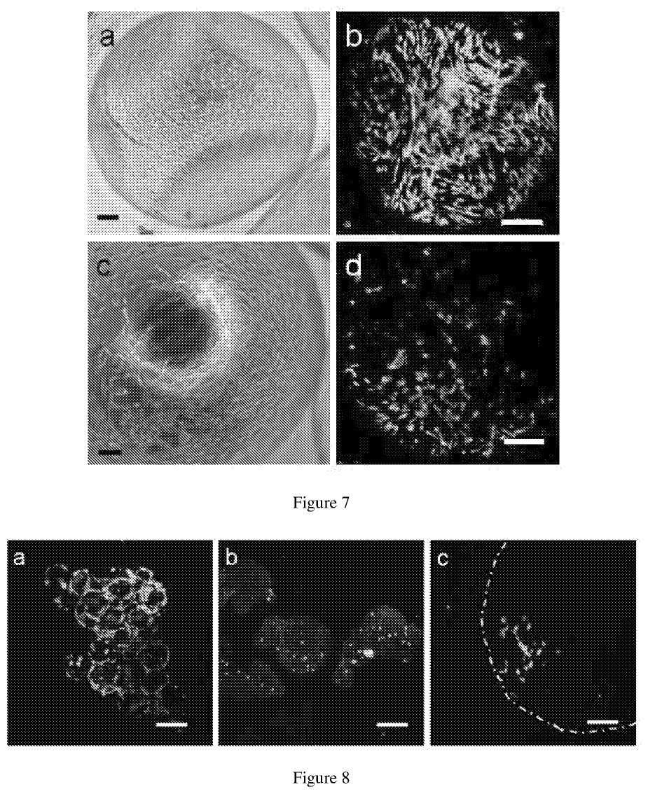

Prior to dynamic culture, microbeads were suspended in a spinner flask containing 20 mL of media and were mechanically stirred for a period of 72 h to check for their shape and integrity. The results showed that the Alg/Gel microbeads provided a microenvironment conducive for cell adhesion (FIG. 7A). Next, to confirm the viability of cells adhered onto the microbeads, a live/dead assay was performed. Results from live/dead assay showed that a vast majority of cells on the fabricated microbeads were viable (FIG. 7 B) which convincingly demonstrates the cytocompatibility of the gelatin-coated alginate beads.

To evaluate the long-term culture of cells on the microbeads, cell-loaded microbeads were cultured under dynamic conditions for 72 h. The cells used for the present Example is obtained by culturing the BM-MSC as per the protocol as described in Example 2. The cultured BM-MSC is further used for expanding as per the microcarrier based method as described in the Example 3. It can be contemplated that BM-MSC obtained commercially can also be used for expanding as per the present protocol.

Subsequently, microbeads were visualized under a phase contrast microscope and a live/dead assay was performed to determine cell adherence, proliferation and viability. Unsurprisingly, the engineered Alg/Gel microbeads demonstrated good stability, surface favorable for cell attachment and negligible cytotoxicity (FIGS. 7 C and 7 D).

Comparative Analysis of the Cell Culture Process Using Alg/Gel Microbeads as Disclosed in the Present Disclosure with Commercially Available Polystyrene (PS) and Recombinant Collagen Peptide (RCP) Beads

The primary purpose of the 3D microcarrier system is to facilitate the adherence of cells and their expansion in a bioreactor setup. Presently, PS and RCP beads are commercially available and have been proved to be efficient in expanding cells in a 3D dynamic culture system. Hence, the fabricated Alg/Gel microbeads as disclosed in the present disclosure were subjected to the same conditions as the other two bead types to get a comparative analysis between all three microcarrier types.

Adherence of cells—The results clearly indicate that the cells adhered significantly to the PS beads as opposed to the other two bead types (FIG. 8). Even though the number of cells that had adhered to RCP (FIG. 8 B) and Alg/Gel microcarriers (FIG. 8 C) were lesser than PS microbeads (FIG. 8A), the viability of cells was found to be unaffected. This indicates that the components used in the preparation of the Alg/Gel beads are cytocompatible and further optimization of these beads would facilitate better adherence of the cells.

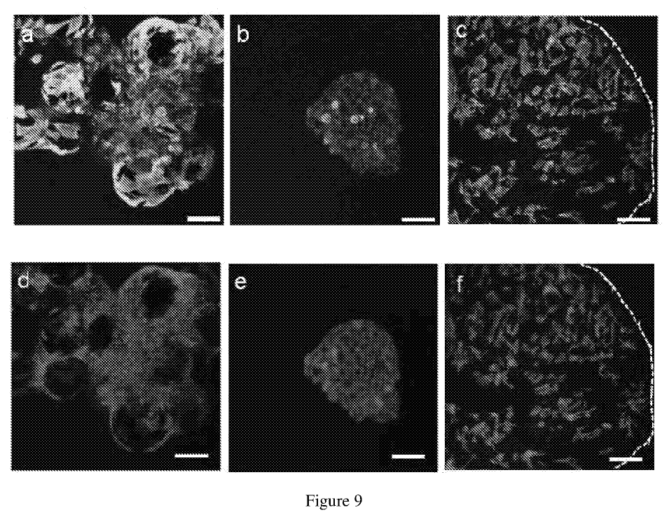

Expression of MSC stemness and stress markers—The expression of alpha smooth muscle actin (aSMA), a stress fiber marker which indicates differentiation to a myofibroblast lineage, was evaluated and compared on cells cultured on all three bead types: PS, RCP and Alg/Gel beads. The results (FIG. 9) show that compared to PS microcarriers (FIG. 9A), RCP (FIG. 9 B) and Alg/Gel microbeads (FIG. 9 C) demonstrated weak expression of aSMA. On the other hand, PS microcarriers (FIG. 9 D) demonstrated better CD90 stem cell marker expression compared to RCP (FIG. 9 E) and Alg/Gel microcarriers (FIG. 9 F).

Decellularization via dissolution of Alg/Gel microbeads—One of the major advantage of the cell culture process using alginate-gelatin microbeads as disclosed in the present disclosure is the ease of recovery of the cultured cells as compared to the available technique in the field. The cells cultured using the microbeads as described herein are amenable to easy recovery by dissolving the microbeads by a protocol as previously described in Example 3. Whereas, such a simple recovery process is not possible by using the PS or RCP beads. In the process using PS or RCP beads, the cells are recovered by decellularization process which is time consuming and a costly affair. Also, the cell-recovery percentage is a concern.

After adherence and expansion of cells on Alg/Gel microcarrier beads, the recovery of cells via minimal manipulation of microbeads and the viability of harvested cells were evaluated. The results are indicative of the fact that the beads were completely dissolved within 10 mins and the viability of the cells (˜80%) was not compromised by the dissolution buffer or by the degraded microbead products (FIG. 10).

### Comparison Matrix Between Alg/Gel Microcarriers, Polystyrene (PS) and Recombinant Collagen Peptide (RCP) Microcarriers—

Table 2 below describes the comparison matrix of the three methods.

TABLE 2

| | | Alg/Gel | | |
|---|---|---|---|---|
| | | microbeads of the | RCP | Polystyrene |
| S. No. | Parameters | present disclosure | microbeads | microbeads |
| 1. | Size distribution (dia, μm) | 340-480 | 100-400 | 125-212 |
| 2. | Bead stability in culture | ++ | +++ | +++ |
| 3. | Dynamic cell loading | ++ | +++ | +++ |
| 4. | Cell viability on beads | +++ | +++ | +++ |
| 5. | Stress biomarkers (αSMA) | low | low | high |
| 6. | Stem cell marker (CD90) | low | low | high |
| 7. | Ease of recovering cells | One-step, Easy | Moderate difficulty | Moderate |
| | | | | difficulty |
| 8. | Weight for cell culture | 1.5 | 3 | 3 |
| | (mg/ml) | | | |
| 9. | No. of microbeads/mg | 50-100 | 500-1000 | 240 |
| 10. | Total cost per gm | $10 | $1700 | $20 |

+++ excellent;
++ good;
+ fair

It can be observed from Table 2 that the microbeads of the present disclosure performs satisfactorily in terms of bead stability and dynamic cell loading. However, in terms of cell viability, expression of stress biomarker and stem cell biomarker the microbeads of the present disclosure performs better than the PS beads. Significant advantages are provides in terms of: (a) ease of cell recovery—it can be observed from Table 2, that the process of cell culturing using microbeads of the present disclosure involves an easy single step of recovering cells, whereas the other process involves moderate to high difficulty; and (b) cost—the present disclosure provides a method which is significantly economical in terms of cost as compared to the other methods.

Table 3 below describes certain non-working examples of cell culturing methods using alginate-gelatin microbeads.

TABLE 3

| | | | Overnight | Bead | Cell |
|---|---|---|---|---|---|
| S. No. | Components | Cross linkers | incubation | integrity | adhesion |
| 1. | Alginate (1-2%) low | Calcium chloride | Sodium | Soft bead | — |
| | viscosity + | (300 mM) | cyanoborohydride | | |
| | Gelatin (1-2%) (1:1) | | | | |
| 2. | Alginate (1-2%) medium | Calcium chloride | Sodium | Stability | No cell |
| | viscosity + | (300 mM) | cyanoborohydride | improved | adhesion |
| | Gelatin (1-2%) (1:1) | | | | |
| 3. | Alginate (1-2%) medium | Calcium chloride | Sodium | Stability | Few cells |
| | viscosity + | (300 mM) | cyanoborohydride | improved | adhered |
| | Gelatin (1-2%) (1:1) | Glutaraldehyde | | | |
| | | (0.4%) | | | |
| 4. | Alginate (1-2%) medium | Calcium chloride | Sodium | Stable upto 3 | Few cells |
| | viscosity + | (300 mM) | cyanoborohydride | days in static | adhered |
| | Gelatin (1-2%) (1:1) | Barium chloride | | | |
| | | (10 mM) | | | |
| | | Glutaraldehyde | | | |
| | | (0.4%) | | | |
| 5. | Alginate (1-2%) medium | Calcium chloride | Water | Stable in | Few cells |
| | viscosity + | (300 mM) | | static | adhered |
| | Gelatin (1-2%) (1:2) | Barium chloride | | Unstable in | |
| | | (10 mM) | | dynamic | |
| | | Glutaraldehyde | | | |
| | | (0.4%) | | | |
| 6. | Alginate (1-2%) medium | Calcium chloride | Calcium chloride | Stable in | Cells |
| | viscosity + | (300 mM) | (100 mM) | static | adhered |
| | Gelatin (1-2%) (1:2) + | Barium chloride | | Unstable in | (>80%) onto |
| | EDTA wash for 20 sec after | (10 mM) | | dynamic | beads in |
| | crosslinking with calcium | Glutaraldehyde | | | static |
| | | (0.4%) | | | |

As per the Table 3, the first non-working example uses low viscosity alginate because of which beads are softer and no cell adhesion can be observed. The second, third, and fourth non-working examples use sodium cyanoborohydride and it was found that cell adhesion and stability is a problem. The fifth non-working example uses water and it can be observed that the beads are not stable under dynamic culture conditions. The sixth non-working example comprises an EDTA wash which was found to provide unstable beads in the dynamic culture. Therefore, the process as disclosed in the present Example is very critical for obtaining the microbeads that can be used to obtain desirable expanded population of mesenchymal stem cells.

### Example 4

### Expanding Stem Cells in Three-Dimensional (3D) Spheroid-Based Culture

### Combination of Hanging Drop and Spinner Flask Methods

The Donor-derived bone-marrow MSC were commercially procured and cultured according to the vendor's instruction.

Initially cells were thawed and cultured in 2D mono-layer in suitable culturing flasks until it reached 90% confluency.

Cells were trypsinized and counted by trypan blue staining.

Cell pellet was resuspended in an appropriate volume of media consisting of either 1:1 ratio of MSC basal media and Methyl cellulose to get 3000 cells/10 μl density or without methyl cellulose.

10 μl drops of cell suspension was added onto the lid of the 96 well plate and wells were filled with 50 μl of sterile 1×PBS for maintaining humidity

After adding the drops the lid was inverted to create hanging drop and plates were incubated at 37° C., 5% CO₂ incubator (static—hanging drop).

Within 16-24 hrs cells were aggregated and formed the spheroids

These spheroids were transferred into spinner flask with either a 1:1 ratio of MSC basal media and methyl cellulose (1%) or without methyl cellulose for dynamic culture condition and incubated at 37° C., 5% CO₂ incubator with magnetic stirring of 115 RPM (dynamic culture in spinner flask).

For control studies spheroids were cultured in MSC basal media without methyl cellulose keeping all the dynamic conditions same

Spheroids were cultured in the same condition for 5 days

Morphology and viability testing were performed by phase contrast imaging and live dead assay respectively on regular time intervals (day 3 and day 5)

On 5^{th} day spheroids were changed with EV-collect media (low serum xeno free medium) and cultured for further 48 hrs keeping all the dynamic conditions same

Morphology and viability testing were performed on 7^{th} day to assess the quality of the spheroids.

### Direct Spinner Flask Method

The Donor-derived bone-marrow MSC were commercially procured and cultured according to the vendor's instruction.

Initially cells were thawed and cultured in 2D mono-layer in suitable culturing flasks until it reached 90% confluency

Cells were trypsinized and counted by trypan blue staining

Cell pellet was resuspended in 15 ml volume of media consisting of 1:1 ratio of MSC basal media and Methyl cellulose to get 3×10⁶ cells in total volume

Cell suspension was transferred into spinner flask with either a 1:1 ratio of MSC basal media and methyl cellulose or without methyl cellulose for dynamic culture condition and incubated at 37° C., 5% CO₂ incubator with magnetic stirring of 90 RPM

For control studies cell suspension was cultured in MSC basal media without methyl cellulose keeping all the dynamic conditions same

Within 24 hrs cells were aggregated and formed the spheroids and allowed to culture in the same condition for 3 days

Morphology and viability testing were performed by phase contrast imaging and live dead assay respectively on regular time intervals

On 3^{rd} day spheroids were changed with EV-collect media (low serum xeno free medium) and cultured for further 48 hrs keeping all the dynamic conditions same.

Morphology and viability testing were performed on 5^{th} day to assess the quality of the spheroids

### Evaluation of Hollow Fiber Bioreactors for the Scale Up Culture of MSCs and Exosome Production

The Hollow fiber bioreactors (HFBs) are a 3D culture system that consist of fibers fixed on a module with cells cultured on the outer surface of porous fibers. The media is then circulated through the fiber capillary lumen, mimicking the in vivo-like circulation of nutrients through blood capillaries. This type of cell culture system allows controlled shear to be applied to cells in culture with dynamic transfer of nutrients and removal of waste products. This creates a versatile cell culture system in which high cell densities can be easily achieved.

A Quantum Cell Expansion System® (Terumo BCT, Colorado, USA) can be used as a part of the present disclosure.

The surface of the hollow fibers is to be coated with human fibronectin (0.05 mg/ml) 18 hours prior to seeding cells, to promote cell adhesion.

The xenofree culture medium is to be equilibrated with a gas mixture (5% O₂, 5% CO₂ and 90% N₂) to provide adequate aeration.

Cells to be seeded at a density of 30×10⁶ cells, (1000 cells/cm²) in the intracapillary space (ICS) for cell adhesion for 24 hours. The cells are to be constantly fed through a continuous flow of culture medium in the extra-capillary space (ECS) with passive removal to waste.

Cells are to be harvested with trypsin as described when a confluency of >90% is reached.

For exosome production, the media is to be replaced entirely with EV Collect (Rooster Bio inc.) and cells is to be cultured for 72 hours. The conditioned media will be collected and harvested as described in the present disclosure.

### Results

### Combination of Hanging Drop & Spinner Flask Methods

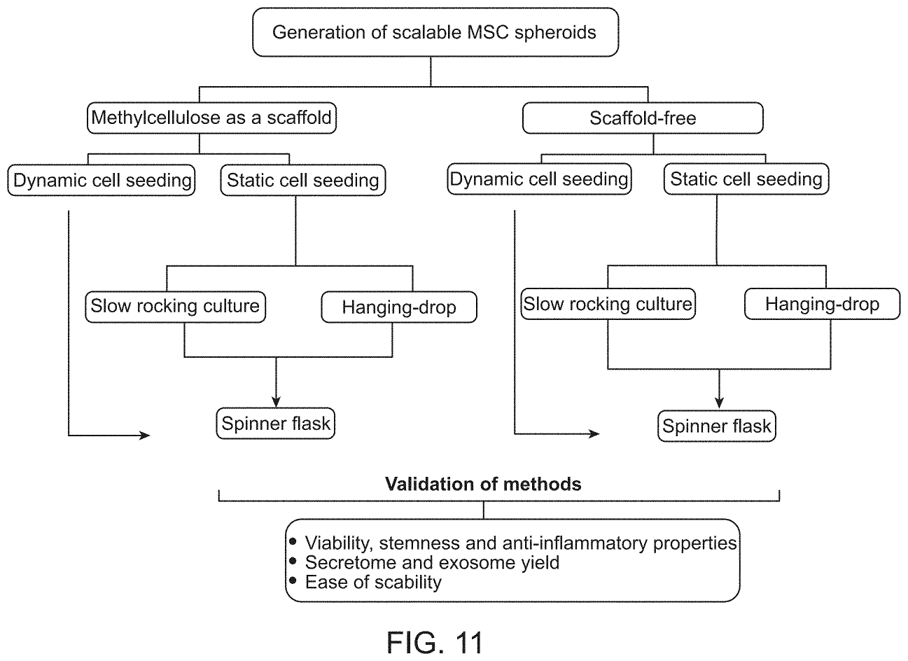

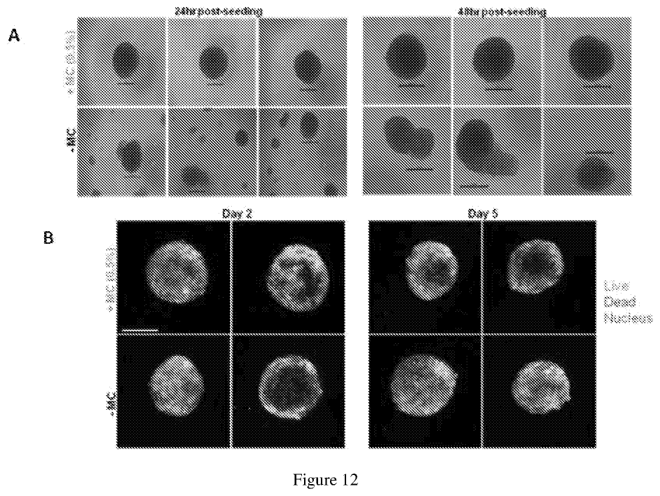

hBMMSC form compact spheroids in the presence of methyl cellulose—A scheme for the production of 3D hBM-MSC spheroids (FIG. 11) and dynamic culture for secretome and exosome production has been disclosed herein. The present data is obtained by culturing BM-MSC. The initial culturing of BM-MSC was done by the protocol explained in Example 2 and the further expansion was done by the present Example. Methyl cellulose was used to enhance the spheroid formation during the hanging drop culture. It was observed that the presence of methyl cellulose enhanced the spheroid forming capacity as evidenced by the single compact cluster of cells, whereas multiple clusters were observed in the hanging drop without methyl cellulose (FIG. 12A). The average size of each spheroid reached up to 200 μm and was maintained throughout the culture period. The spheroids without methylcellulose showed multiple clusters of cells even after 48 h post seeding. Viability staining performed on spheroids collected on day 2 and day 5 did not show a significant difference in the viability of cells in the presence of methyl cellulose when compared with the spheroids without methylcellulose. These results suggest that presence of methyl cellulose in the hanging drops reduces the spheroid forming time without affecting the viability of the cells, possibly due to the increased viscosity of the culture medium with the methyl cellulose.

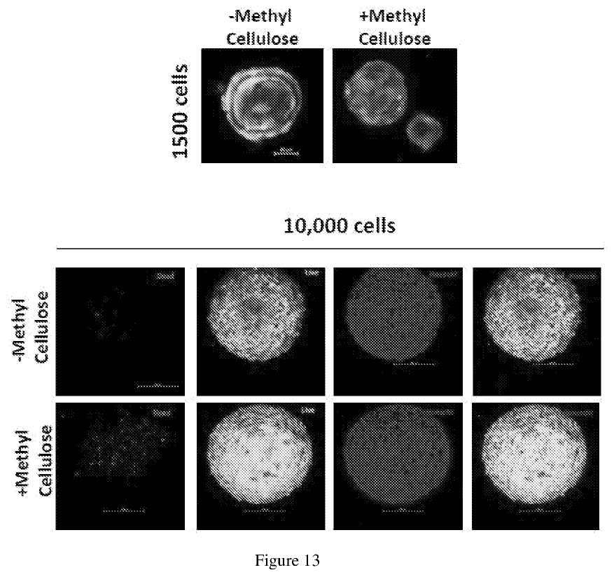

Spheroid formation at a lesser cell density of 1500 cells and higher cell density of 10,000 cells per spheroid using the hanging drop method was also demonstrated. It was found that 1500 cells produced smaller spheroids (50-100 μm) (FIG. 13A), comparable sizes in the presence and absence of methyl cellulose while seeding at a higher density of 10,000 cells resulted in the formation of spheroids of approximately 200 μm in the absence of methyl cellulose and 200-300 μm in the presence of methyl cellulose in 24-72 hours (FIG. 13 B). As also shown in FIG. 13 B, increased cytotoxicity (dead cells) at this seeding density was also observed. Interestingly, increased cytotoxicity in spheroids plus methyl cellulose was observed compared to the spheroids without methyl cellulose (FIG. 12-13). Hence, a range is provided for the concentration of methyl cellulose that can be used in this protocol in Table 4.

TABLE 4

| SI. no | Parameters | Working range | |
|---|---|---|---|
| 1 | Initial seeding density (cells/spheroid) | 600-10,000 | |
| 2 | Conc. of Methylcellulose | 0.2-2% | |
| 3 | Cell aggregation time in hanging drop | 6 h-24 h | |
| 4 | Spheroid maturation time in dynamic culture | 3-7 | days |
| 5 | Time window for exosome collection (post- | Day 3-Day 7 | |
| | maturation) | | |
| 6 | Diameter of the spheroids | Hanging drop: 100-300 μm | |
| | | Direct spinner flask: 30-250 μm | |
| 7 | Speed of the magnetic stirrer | 50-150 | rpm |

Combination of Slow Rocking Culture Step with Spinner Flask Dynamic Culture of Spheroids

An alternate hanging drop protocol can be adopted in which the spheroid formation+/−methyl cellulose occurs on a rocking platform instead of in a hanging drop. The critical step (when compared to the technique known in the art) would be the presence of methyl cellulose in the culture medium to allow compact and rapid spheroid formation.

A 1-4 tier, multi-shelf rocker system can be placed inside an incubator at 37° C. during spheroid production. The spheroids will have continuous supply of 95% oxygen, 5% carbon dioxide gas mixture. The culture will be maintained at a rocking speed of 10-30 cycles/min with a 5-10° range of motion. Spheroids will be allowed to form at the same seeding density described in Table 4 in the presence of methyl cellulose.

hBM-MSC spheroids shows enhanced protein secretion in the dynamic culture—To address the challenges faced on obtaining the sufficient number of exosomes produced using the conventional monolayer culture; the efficiency of MSC spheroids in terms of production of quality and quantity of secretome, which includes some of the therapeutically important factors such as HGF, NGF, etc was evaluated.

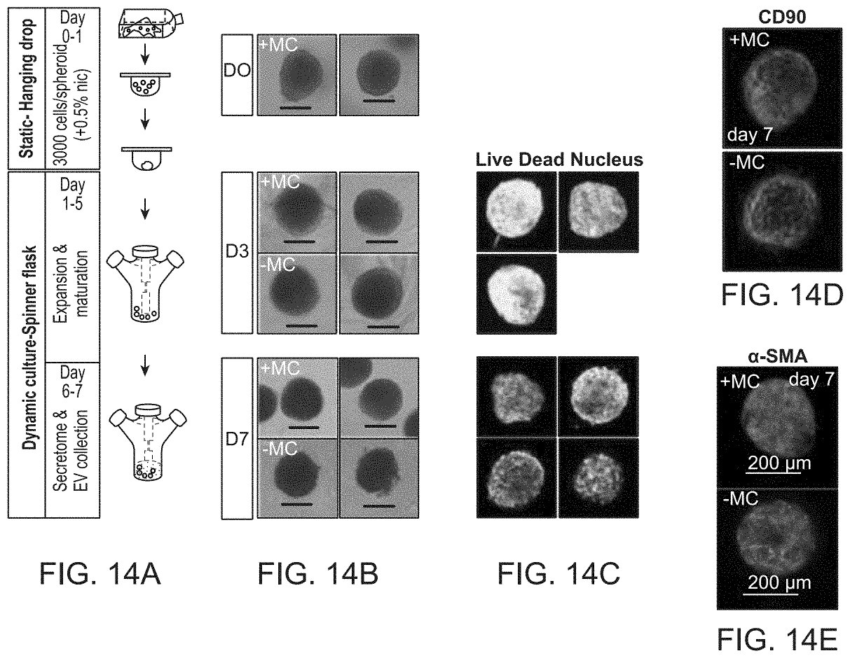

After forming the compact 3D spheroids of hBM-MSC by hanging drop culture, the spheroids were introduced into the dynamic system using spinner flask with and without methyl cellulose. FIG. 14A, depicts the scheme of the experiment whereby spheroids formed by the static hanging-drop culture in the presence of 0.5% methyl cellulose and having a density of 3000 cells per spheroid were introduced into the dynamic culture for secretome or exosome production.

A control culture was kept without the presence of methyl cellulose in the dynamic culture system. Consistent and compact spheroids were observed in the dynamic culture throughout the culture period in both with and without methyl cellulose (FIG. 14B). Live-dead staining performed on the spheroids from day 3 and day 7 showed a significant number of viable cells (FIG. 14C). The expression of CD90 (stemness marker) (FIG. 14D) and α-SMA (stress marker) (FIG. 14E) pattern was checked after 7 days in the dynamic culture. It was observed that the CD90 expression was maintained in the dynamic culture indicating that MSC maintained their stem cell properties while low expression of α-SMA was detected in the spheroids.

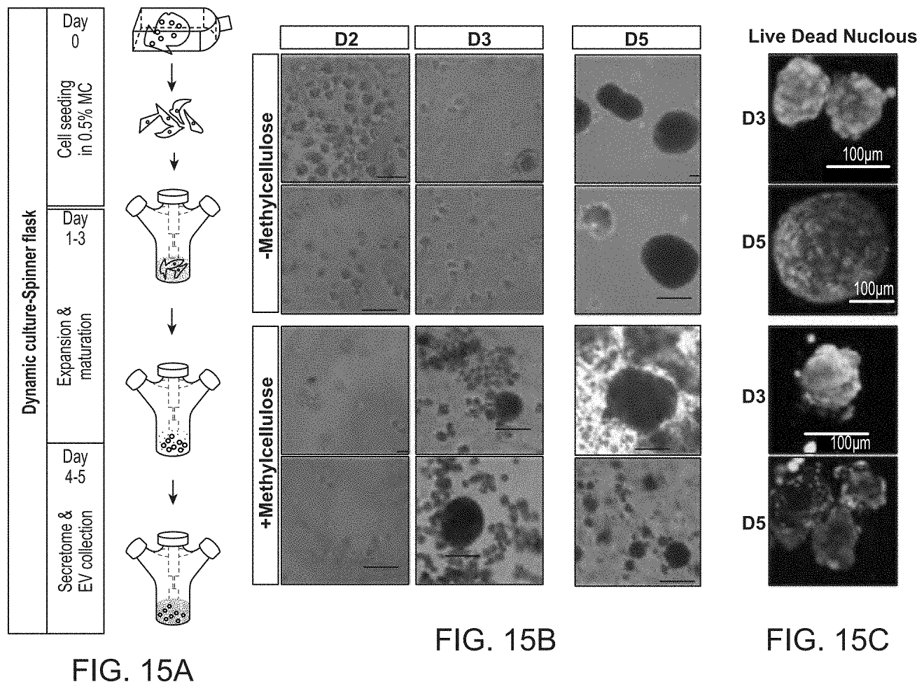

Direct spinner flask method—Besides all the efforts in scaling up MSC culture for cell and exosome therapy. There is also a growing interest in enhancing their therapeutic potential by providing the 3D culture conditions. In this regard, the use of bioreactors such as spinner flasks, rotating wall vessels and hollow fiber bioreactors have been utilized to provide a dynamic culture conditions that will increase the oxygen and nutrients supply to cells and the removal of waste products and produce fluid shear stress, which confer biomechanical cues that are the important aspect of the cellular environment and can alter the properties and behavior of cells. In this alternative method, we demonstrate the direct 3D spheroid culture by seeding the cells with a polymer-based support in a spinner flask. Unlike the published methods of spheroid generation, that require very high density of cells (˜1 million cells per ml), our method requires 5-fold lesser cell density (0.2 million cells/ml) for the spheroid formation. When cultured with 0.75% methyl cellulose, the cell formed predominantly uniform clusters ranging from 30-100 μm (FIG. 15 B). It was found that these clusters were stable and showed viable cells during the culture period (FIG. 15 C). In the absence of methyl cellulose, the spheroids were observed in the spinner flask, however the efficiency of the cell aggregation was lower as evidenced by the settlement of cells to the bottom of flask. Moreover, at day 5 more number of cell aggregates was observed in the spheroid cultured with methyl cellulose compared with the spheroids cultured without methylcellulose.

It can be appreciated from Examples 3 and 4 that the stem cells cultured by the method as described herein shows the desirable expansion in terms of number as well as in terms of its stemness and other desired characteristics. Although a specific example of human bone marrow derived mesenchymal stem cells has been described herein, however, it can be contemplated that all other types of stem cells in general and mesenchymal stem cells in particular can be cultured by the process as described herein.

### Example 5

Isolation and Purification of Secretome and Exosomes from the Cell Culture

The conditioned medium was collected from the CSSC and hBMMSC according to the process as described in Example 1 and 2, respectively. The obtained conditioned medium was directly used as secretome or subjected to ultracentrifugation for isolating exosomes. Isolation of exosome from secretome was done by using three methods: (i) Single step ultracentrifugation; (ii) Sucrose based cushion density ultracentrifugation and (iii) Iodixanol density gradient ultracentrifugation. All of the three methods followed a second round of purification using size exclusion chromatography (using Captocore 700 column). Capto Core 700 is composed of a ligand-activated core and inactive shell. The inactive shell excludes large molecules (cut off ˜Mr 700 000) from entering the core through the pores of the shell. These larger molecules are collected in the column flow through while smaller impurities bind to the internalized ligands. Furthermore, the resin Captocore700 is scalable to a capacity in litres.

The detailed process of each purification method is explained below:

(I) Single-Step Ultracentrifugation:

The following steps were followed to purify the exosomes using single-step centrifugation:
(i) Once the cells reached 80-90% confluency, the media was removed, and cells were washed in 1× Phosphate-Buffered-Saline (PBS) (20 ml). PBS was discarded and 260 mL of extracellular vesicles (EV) collect media was added to the flasks and the flasks were then incubated for 72 h at 37° C., 5% CO₂.
(ii) The supernatant was collected and immediately proceeded with the pre-processing steps as described below:
   The media was centrifuged at 300×g for 10 min at 4° C., and the supernatant was collected.
   The supernatant was centrifuged at 3000×g for 20 min at 4° C. and the supernatant was collected.
   The supernatant was centrifuged at 13000×g for 30 min at 4° C. and the supernatant was collected.
   The media was then filtered through a 0.45-micron filter.
   The media was further filtered through a 0.22-micron filter.
(iii) The conditioned media was stored at 4° C. for short term storage (24 h) or at a temperature of −80° C. for long term storage (1 month).
(iv) Enrichment of exosomes pellet by ultracentrifugation: To process the cells immediately, following processing steps were followed. In case of frozen cells, the conditioned media was thawed at 4° C. prior to execution of the steps described below:
   The conditioned media was centrifuged at 100,000×g for 90 min at 4° C.
   The supernatant was removed carefully, and a clear pellet was observed at the bottom of the tube.
   The final centrifugation was done by dissolving the pellet in either PBS, or Plasma-Lyte A, or Saline. About 0.5 m of crude exosomes were stored at −80° C. for QC.

(II) Sucrose-Based Cushion Density Ultracentrifugation:

The following steps were followed to purify the exosomes using sucrose-based cushion density centrifugation:
(i) Once the cells reached 80-90% confluency, the media was removed, and the cells were washed in 1× Phosphate-Buffered-Saline (PBS) (20 ml). PBS was discarded and 260 mL of extracellular vesicles (EV) collect media was added to the flasks and the flasks were then incubated for 72 h at 37° C., 5% CO₂.
(ii) The supernatant was collected and immediately proceeded with the pre-processing steps as described below:
   The media was centrifuged at 300×g for 10 min at 4° C., and the supernatant was collected.
   The supernatant was centrifuged at 3000×g for 20 min at 4° C. and the supernatant was collected.
   The supernatant was centrifuged at 13000×g for 30 min at 4° C. and the supernatant was collected.
   The media was then filtered through a 0.45-micron filter.
   The media was further filtered through a 0.22-micron filter.
(iii) The conditioned media was stored at 4° C. for short term storage (24 h) or at a temperature of −80° C. for long term storage (1 month).
(iv) Enrichment of exosomes pellet by ultracentrifugation: To process the cells immediately, following processing steps were followed. In case of frozen cells, the conditioned media was thawed at 4° C. prior to execution of the steps described below:
(v) The conditioned media was centrifuged at 100,000×g for 90 min at 4° C.
(vi) The supernatant was removed carefully, and a clear pellet was observed at the bottom of the tube.
(vii) Purification of the enriched exosomes by 30% sucrose density ultracentrifugation:
   The enriched exosomes were transferred on to 30% sucrose (1M) containing ultracentrifuge tube (according to the process as described in Gupta, S., Rawat, S., Arora, V. et al. An improvised one-step sucrose cushion ultracentrifugation method for exosome isolation from culture supernatants of mesenchymal stem cells. Stem Cell Res Ther 9, 180 (2018). https://doi.org/10.1186/s13287-018-0923-0).
   The ultracentrifuge tube was spun at a speed of 1000000 g for 2 hr at 40 C, and the acceleration and deceleration were set to zero.
   The supernatant was carefully removed, and the exosomes were resuspended in sterile 1×PBS, in order to remove the sucrose and to obtain the exosomes in pellet.
   The exosomes were aliquoted and were stored at store at −80° C.

(III) Iodixanol Density Gradient Ultracentrifugation:

The following steps were followed to purify the exosomes using iodixanol cushion density centrifugation:
(i) Once the cells reached 80-90% confluency, the media was removed, and the cells were washed in 1× Phosphate-Buffered-Saline (PBS) (20 ml). PBS was discarded and 260 mL of extracellular vesicles (EV) collect media was added to the flasks and the flasks were then incubated for 72 h at 37° C., 5% CO₂.
(ii) The supernatant was collected and immediately proceeded with the pre-processing steps as described below:
   The media was centrifuged at 300×g for 10 min at 4° C., and the supernatant was collected.
   The supernatant was centrifuged at 3000×g for 20 min at 4° C. and the supernatant was collected.
   The supernatant was centrifuged at 13000×g for 30 min at 4° C. and the supernatant was collected.
   The media was then filtered through a 0.45-micron filter.
   The media was further filtered through a 0.22-micron filter.
(iii) The conditioned media was stored at 4° C. for short term storage (24 h) or at a temperature of −80° C. for long term storage (1 month).
(iv) Enrichment of exosomes pellet by ultracentrifugation: To process the cells immediately, following processing steps were followed. In case of frozen cells, the conditioned media was thawed at 4° C. prior to execution of the steps described below:
The conditioned media was centrifuged at 100,000×g for 90 min at 4° C.
The supernatant was removed carefully, and a clear pellet was observed at the bottom of the tube.
The pellet was dissolved in 36 mL EV collect media (36 mL per 300 mL starting conditioned media). About 0.5 m of crude exosomes were stored at −80° C. for QC.
(v) Density gradient ultracentrifugation (DGUC):
   An Iodaxinol (IDX) gradient fractions were prepared by floating 3 ml of 10% w/v IDX solution ((Sigma #D1556) containing NaCl (150 mM) and 25 mM Tris:HCl (pH 7.4), over 3 ml of 55% w/v IDX solution.
   The conditioned Media (6 ml) was floated on the top of the IDX cushion and was then allowed to ultracentrifuge by using a Beckman Coulter SW 40 Ti rotor for 4.5 hours at 100,000×g (4° C.).
   Twelve IDX gradient fractions (1 ml each) were collected from the top of the gradient. Fraction collection was carried out on ice and each fraction was collected into pre-chilled 1.5 ml tubes.
   About 9 IDX gradient fractions were transferred into a fresh ultracentrifuge tube and 11 ml PBS was added to the 1 mL fraction. The ultracentrifugation step was repeated at 100,000×g for 4 h in Optima XPN-100 ultracentrifuge using a Beckman Coulter SW 40 Ti rotor at 4° C.
   The supernatant was discarded. The exosome pellet was then resuspended in 1 ml PBS.
   About 50-100 μL aliquots of exosomes were stored at 4° C. for short term (2-3 days) and −80° C. for long term storage.

(IV) Purification of Exosomes by Size Exclusion Chromatography Using CaptoCore 700 Column:

Exosomes isolated by the above three methods (I, III, and III) were further purified by running through a size exclusion chromatography column—1 ml (CaptoCore 700, GE). The steps are described below:
(v) A 1 ml column was equilibrated with 1×PBS (5 times). Post equilibration, the exosome sample (50-100 μl) was loaded into the column.
(vi) The exosomes were eluted in a total of 1 mL 1×PBS/PlasmaLyte A/Saline in fractions of 35 μL (approximately 26 fractions). About 2-3 fractions contained exosomes. The fractions were then manually collected into 1.5 ml tubes kept on ice.

The tubes containing purified fractions of exosomes were stored at 4° C. for short term (2-3 days) and −80° C. for long term storage.

### Results

Characterization of the secretome obtained from the conditioned medium collected by the 3D culture methods as described in the Examples 3 and 4.

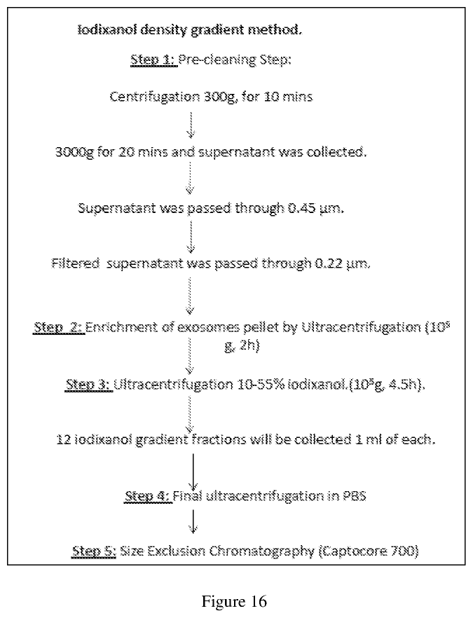

The conditioned medium was collected from the CSSC and hBMMSC 2D cultures as described in the Examples 1 and 2. The obtained conditioned medium was directly used as secretome or subjected to ultracentrifugation for isolating exosomes. Isolation of exosome from secretome was done using Iodixanol density gradient ultracentrifugation (FIG. 16). The purified exosomes were further characterized using multiple methods like the Nano tracking analysis (NTA), transmission electron microscopy (TEM) and western blot.

Characterization of Secretome from BM-MSC and CSSC Cultured by Two-Dimensional Culture Methods

The respective cells were obtained by the methods as described in Example 2 and 1, respectively.

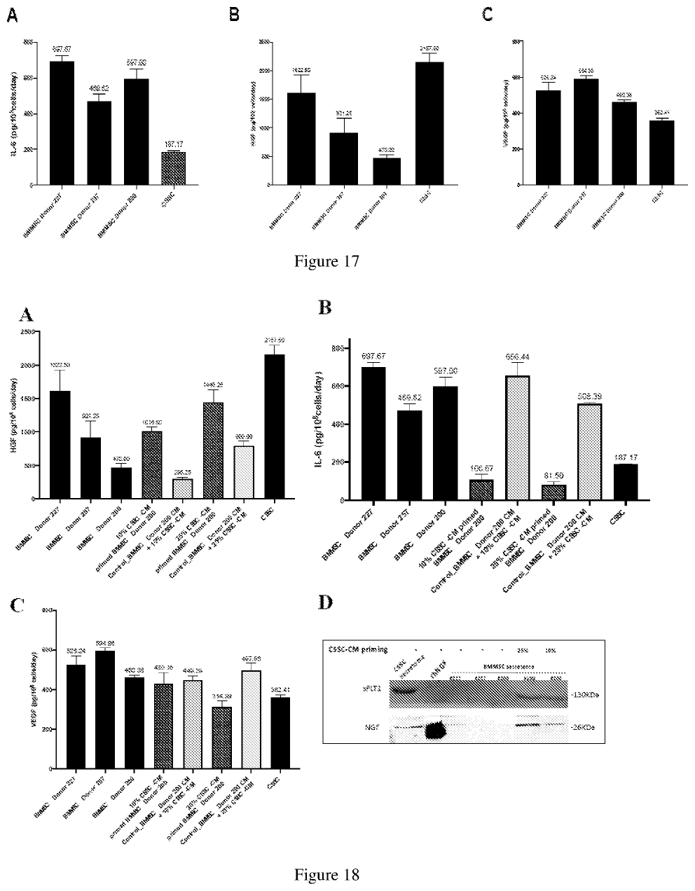

The secretome of BMMSCs from three independent donors (#200, #227, #257) were harvested alongside CSSCs and secreted levels of VEGF, HGF and IL-6 were quantified. CSSCs were found to secrete significantly lower levels of pro-inflammatory IL-6 compared to BMMSCs while priming of BMMSCs with CSSC-conditioned medium resulted in a marked decrease in the level of IL-6 secreted by the primed BMMSCs (FIG. 17A). BMMSCs from all three donors were found to secrete more VEGF than CSSCs (FIG. 17 C), while CSSCs were observed to express more HGF levels (FIG. 17 B).

### BMMSCs Cultured in 3D as Spheroids as Compared to 2D Culturing Methods

The MSC (hBM-MSC) were cultured as per the method described in the Example 4 for 3D spheroid-based culturing, and as per the Example 2 for 2D based culturing.

The protein content in the secretome obtained from the conditioned medium in 3D spheroids and 2D methods was quantified by Bradford method. The amount of protein was normalised to per millions of cells and presented as protein yield per million cells per day. A differential amount of protein was found to be present in the secretome of 2D and 3D samples. When compared with 2D hBM-MSC, which were incubated in secretome collection medium, a 4.8-folds and 3.2-folds more protein in 3D spheroids cultured with and without methyl cellulose respectively, was observed. The increase in the protein content may directly correlate with the amount of therapeutically important factors present in the secretome (Table 5). Table 6 depicts the cell viability, biomarker expression levels, and total secreted protein. Thus, it can be inferred that 3D culturing methods as described in the Examples 3 and 4 are a viable option to scale-up MSC-exosome production in order to meet the current challenges faced in obtaining therapeutic dose of exosome which is cost-effective, consistent and less labor intensive.

TABLE 5

| | | | Fold increase in |
|---|---|---|---|
| | Cell Number | Total protein secreted/ | protein compared |
| Cells Types | (million) | million cells (mg)/day | to 2D |
| 2D hBM-MSC | 6.5 | 0.045 | — |
| 3D hBM-MSC Hanging drop + | 125 spheroids (0.375) | 0.217 | 4.8 |
| Spinner flask (+methyl | | | |
| cellulose) | | | |
| 3D hBM-MSC Hanging drop + | 125 spheroids (0.375) | 0.145 | 3.2 |
| Spinner flask | | | |
| (−methyl cellulose) | | | |

TABLE 6

| | | 2D cultures (as | Microcarrier culture | Spheroid culture (as |
|---|---|---|---|---|
| | | per Example 2; | (as per Example 6; | per Example 7; |
| S. No. | Parameters | hBM-MSC) | hBM-MSC) | hBM-MSC) |
| 1. | Cell viability | >98% | >90% | >90% |
| 2 | Biomarker | High expression of | Moderate expression of | High expression of |
| | expression | CD90 | CD90 | CD90 |
| 3. | Total secreted | 0.045 | — | 0.145-0.217 |
| | protein (mg/million | | | (3- to 5-fold increase) |
| | cells/day | | | |

Characterization of Purified Exosomes from MSCs (2D Culture):

The conditioned medium was collected from the CSSC and hBMMSC 2D cultures as described above (Example 1 and 2, respectively). The obtained conditioned medium was directly used as secretome or subjected to ultracentrifugation for isolating exosomes. Isolation of exosome from secretome was done using three methods namely (i) Single step ultracentrifugation; (ii) Sucrose based cushion density ultracentrifugation and (iii) Iodixanol density gradient ultracentrifugation. The three protocols will be followed by a second round of purification using size exclusion chromatography (CAPTOCORE 700).

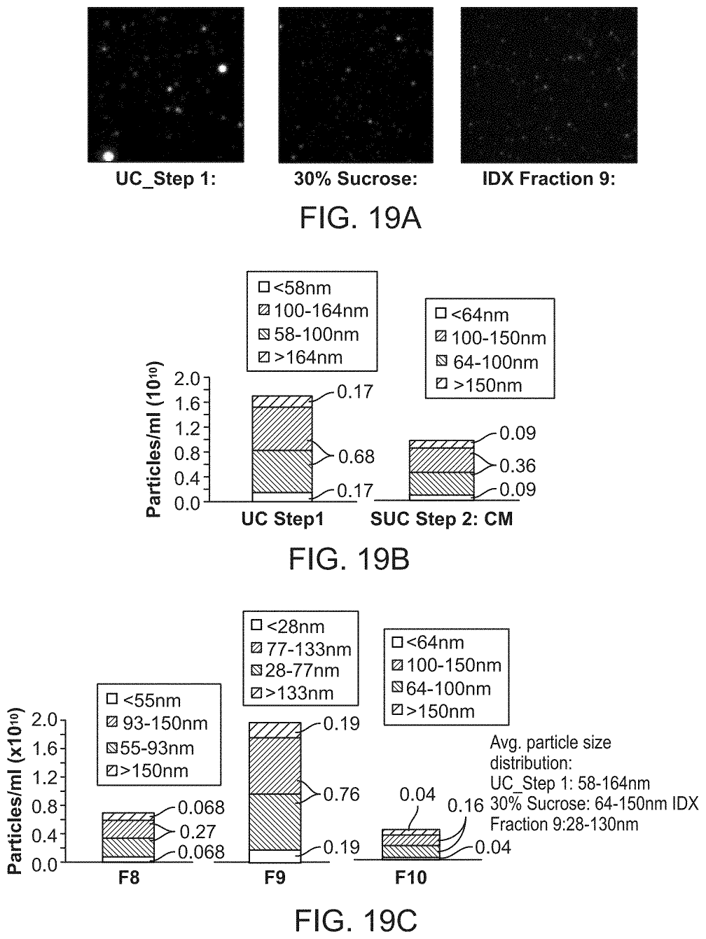

The purity of exosomes isolated by the methods is the key differentiating factor between the protocols: Iodixanol protocol (highest purity)>30% sucrose cushion>single step ultracentrifugation (lowest purity) (see FIGS. 19A-19C). Comparison of exosome population isolated by Single step ultracentrifugation (UC_Step1), 30% sucrose cushion and iodixanol gradient ultracentrifugation protocols: FIGS. 19A-19C demonstrate the heterogeneity of the exosome particle size obtained in each method of purification. Single step UC purification of exosomes results in isolation of particles in the range of 50-170 nm, 30% sucrose cushion gives us particles in the range of 60-150 nm while iodixanol gives us a tighter range of 30-130 nm. Particle heterogeneity: Single step UC>30% sucrose cushion >iodixanol gradient method. UC_Step1: single step ultracentrifugation; SUC Step2:CM: 30% Sucrose cushion ultracentrifugation; IDX F8-F10: Iodixanol density gradient ultracentrifugation fractions 8, 9, 10.

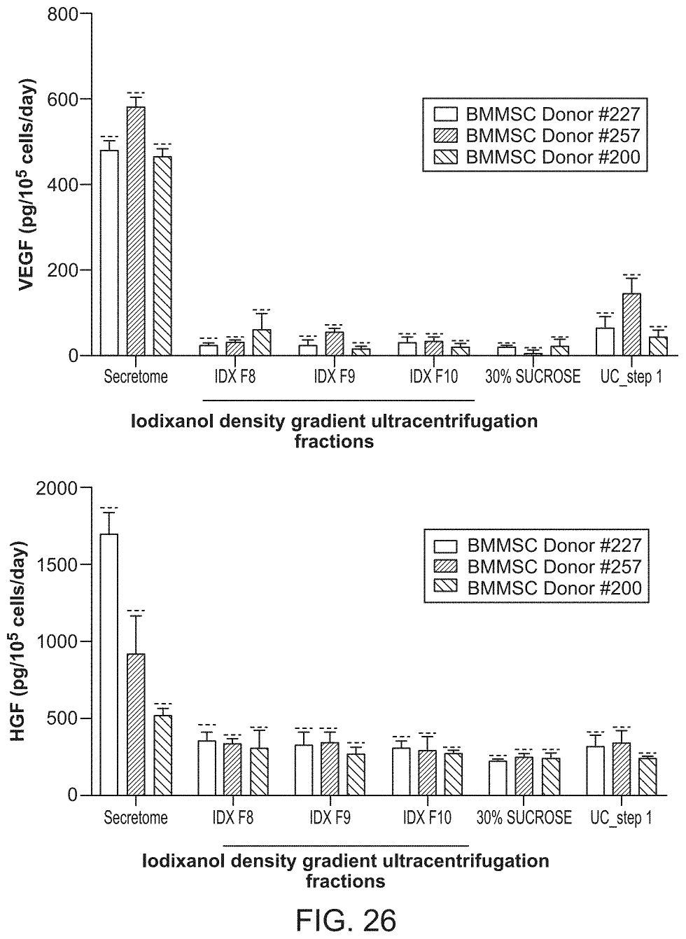

Capto Core 700 is composed of a ligand-activated core and inactive shell. The inactive shell excludes large molecules (cut off ˜Mr 700 000) from entering the core through the pores of the shell. These larger molecules are collected in the column flow through while smaller impurities bind to the internalized ligands. Furthermore, the resin Captocore700 is scalable to a capacity in litres. Exosomes of different purities will be developed for target indication specificity. For example, a combination of iodixanol density gradient Ultracentrifugation or 30% sucrose cushion+Captocore700 would give the highest purity with minimal contamination with angiogenic factors (e.g. VEGF) that would be ideal for application in avascular tissues such as cornea (FIG. 26). A less rigorous purification protocol such as 30% sucrose or iodixanol density gradient ultracentrifugation only protocol would be useful in the treatment of vascular tissue related diseases where the presence of angiogenic factors would not bear any adverse effects e.g. ARDS (lung).

The purified exosomes were further characterized using multiple methods like the Nano tracking analysis (NTA), transmission electron microscopy (TEM), western blot and ELISA based immune assays.

### Working Example 1: Characterization of Purified Exosomes from BMMSCs by Iodixanol Gradient Ultracentrifugation

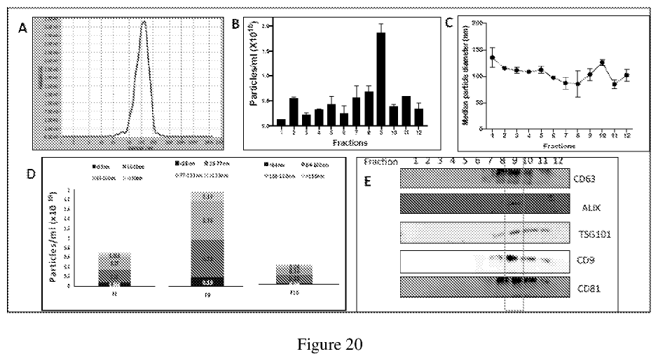

Characterization of hBM-MSC derived exosomes: Conditioned media was processed by density gradient ultracentrifugation. A total of 12 fractions were collected and characterized by nanoparticle tracking analysis (NTA, quantitative) and western blot (qualitative) (FIG. 20).

FIG. 20A-B depicts the particle concentration of fraction 9 (F9): 1.8×10¹⁰/ml); C. Median particle diameter in nm ranged between 100-150 nm; D. Avg. size distribution of F9: 28-133 nm. Particle size distribution and particle number were determined by NTA. Particles were detected at 11 different positions of the cell and averaged. Each sample was run in 3 technical replicates. E. Exosomes (fraction 9) isolated from hBM-MSCs were positive for typical exosome markers including CD63, CD9, CD81, ALIX and TSG101.

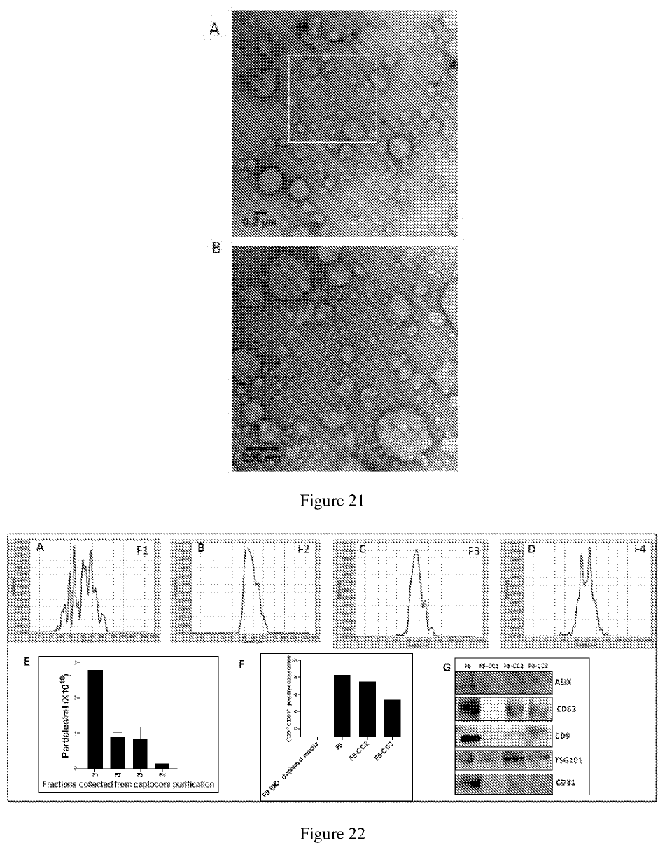

FIG. 21 depicts the Transmission Electron Microscopy (TEM) images of exosomes isolated by iodixanol density gradient ultracentrifugation. Lower magnification of representative images is shown in (A) and the respective magnified image (marked in yellow box) is shown in (B). Scale bars (0.2 μm (E), and 200 nm (F)). The TEM images shows exosomes in the expected size range of about 150-250 nm range and complements the NTA data.

### Working Example 2: Characterization of Purified Exosomes from BMMSCs Purified by a Combination of Iodixanol Gradient Ultracentrifugation and Size Exclusion Chromatography

Purification of exosomes by size exclusion chromatography: Column was equilibrated with PBS 5 times. The sample (100 ul of F9) was loaded and eluted in 1 ml of PBS (as per reference) in 35 μl fractions (26 fractions). Eluted subfractions 2 & 3 were found to contain maximum yield of exosomes. The exosome profile, size distribution and protein cargo were also characterized (FIG. 22). The fractions 2&3 were pooled and tested in functional assays (hereafter referred as F9-CC). FIG. 22 depicts the Exosome size distribution and cargo characterization post size exclusion chromatography. (A-D) All fractions up to F7 were run on NTA. From F5, no particles were detected and only alternate fractions were run thereon. (E) Particle concentration per fraction (Fraction 9 was diluted into two fractions (2+3). (F) Flow cytometry analysis of fraction 2 and 3 from captocore purification identified 75% and 54% of the exosome population in fraction 2 and fraction 3 to be CD81/CD9 positive, respectively. (G) Western blot analysis of exosome markers CD81, CD9, CD63, ALIX and TSG101 in captocore purified fraction 9.

### Working Example 3: Characterization of Purified Exosomes from BMMSCs Purified 30% Sucrose Cushion Ultracentrifugation

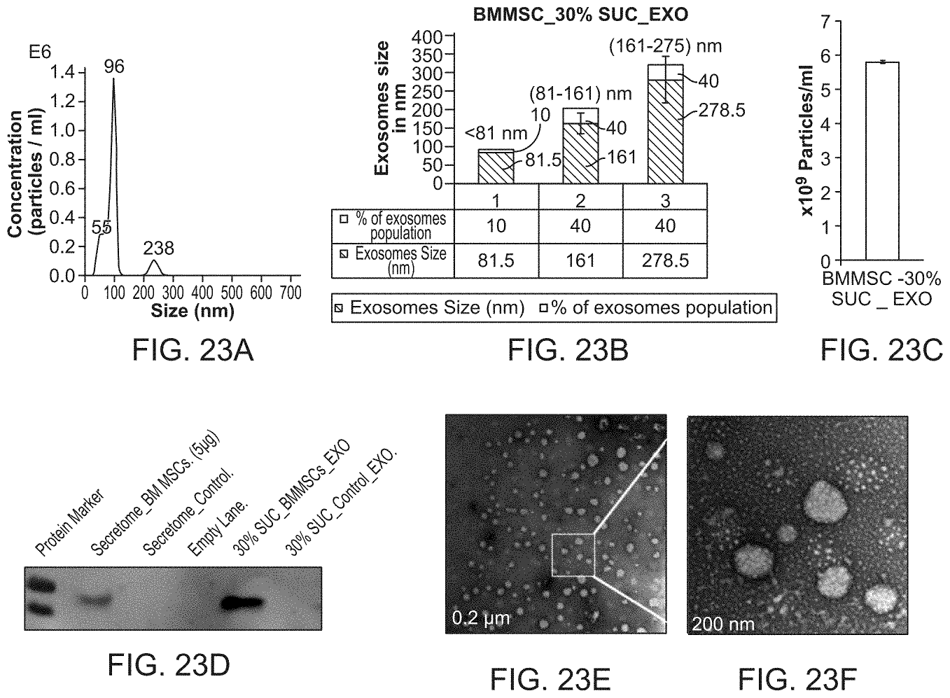

The 30% sucrose cushion density ultracentrifugation yielded higher particle numbers compared to iodixanol (approximately 5 folds higher) (FIGS. 23A-23C). However, the particle size distribution was more heterogenous with roughly 40% of the exosomes falling in the size range of >150 nm (161-275 nm) (FIG. 23B).

The exosomes were found to express CD9, a key exosome marker and maintained their integrity/morphology in solution as shown in FIG. 23D and FIGS. 23E-23F respectively. FIGS. 23A-23C depicts Size distribution analysis of exosomes purified from BMMSCs by 30% cushion-based sucrose density method using Nano Tracking Analysis (NTA). A representative image of histogram is shown in A. The averaged data from 3 independent readings of size distribution are presented in FIG. 23B. FIG. 23C shows the total yield of exosomes from 30% sucrose cushion ultracentrifugation determined by NTA. FIG. 23D shows Western blot analysis for exosome marker CD9. Protein samples from secretome and exosome preparation were separated on a 12% SDS PAGE gel and antibody against CD9 was used to identify exosomes. CD9 was present both in secretome and exosome samples showing expected size of 24-27 Kda and the control samples were negative. FIGS. 23E and 23F show Transmission Electron Microscopy (TEM) images of exosomes isolated by 30% sucrose method. Lower magnification of representative images is shown in FIG. 23E and the respective magnified image (marked in yellow box) is shown in FIG. 23F. Scale bars (0.2 μm (FIG. 23E), and 200 nm (FIG. 23F)). The TEM images shows exosomes in the expected size range of about 150-250 nm range and complements the NTA data.

### Working Example 4: Characterization of Purified Exosomes from CSSCs Purified by Sucrose Cushion Ultracentrifugation and Iodixanol Density Gradient Ultracentrifugation

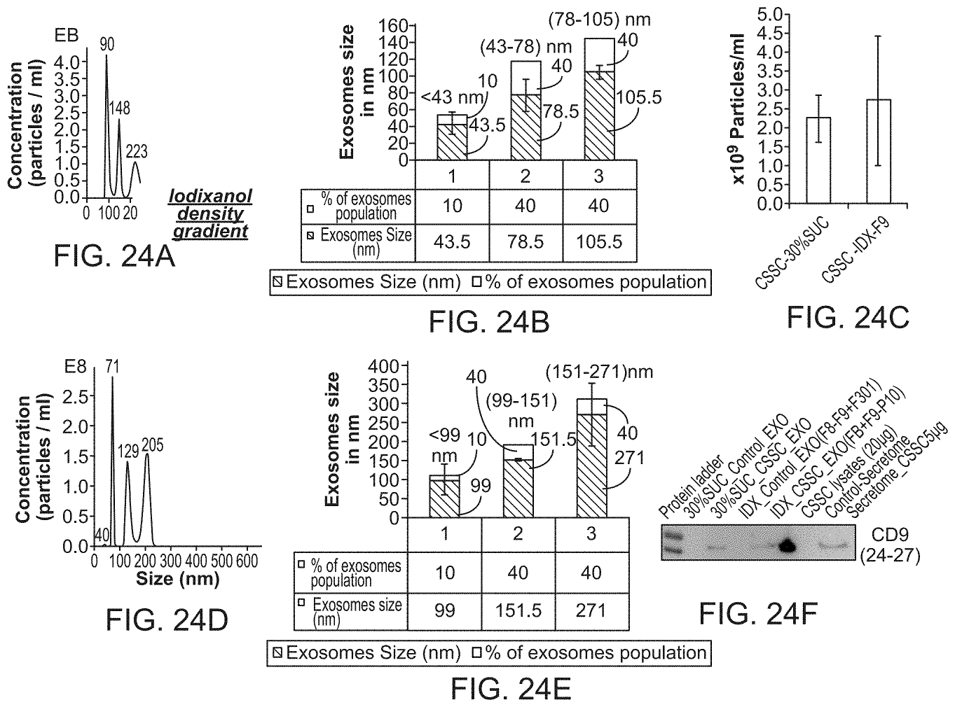

Exosomes from CSSCs isolated by both 30% sucrose cushion (FIGS. 24A-24B) and Iodixanol density gradient ultracentrifugation (FIGS. 24E-24F) were more heterogenous compared to BMMSC derived exosomes. However, the particle numbers isolated by iodixanol gradient was comparable to the exosome yield from BMMSCs (FIG. 24C). The exosomes isolated by both methods expressed similar levels of exosomal marker CD9 (FIG. 24F). FIGS. 24A-24E depicts Size distribution analysis of exosomes purified from CSSCs by 30% sucrose cushion density (30% SUC) based ultracentrifugation (FIGS. 24A-24C) and iodixanol density gradient ultracentrifugation (IDX Fraction 9 (IDX-F9)) method (FIGS. 24D-24E) using Nano Tracking Analysis (NTA). A representative image of histogram is shown in FIGS. 24A and 24D for 30% SUC and IDX-F9, respectively. The averaged data from 3 independent readings of size distribution are presented in B &E for 30% SUC and IDX-F9 respectively. FIG. 23C shows the total yield of exosomes from 30% SUC and IDX-F9 respectively determined by NTA. FIG. 24F shows Western blot analysis for exosome marker CD9 for 30% SUC and IDX-F9. Protein samples from secretome and exosome preparation were separated on a 12% SDS PAGE gel and antibody against CD9 was used to identify exosomes. CD9 was present both in secretome and exosome samples showing expected size of 24-27 Kda and the control samples were negative.

### Reproducibility of the Protocol of Exosome Production (2D Culture) of the Present Disclosure:

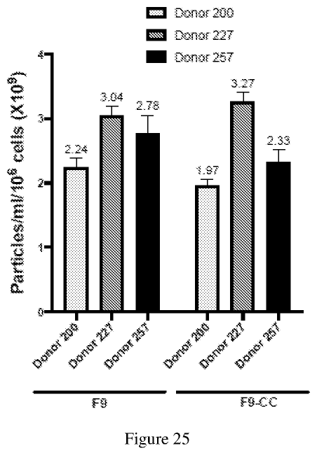

Three independent donors of hBMMSCs were expanded using the 2D protocol described above. Cells were expanded in xenofree culture medium and exosomes were collected post 72 h incubation in RoosterBio EV Collect media. Exosomes were purified by Iodixanol density gradient ultracentrifugation from a total volume of 200 ml per donor. Fraction 9 was collected (F9) and half of the fraction was further purified by size exclusion chromatography (F9-CC). With the present protocol of the disclosure an average of 2.7×109±0.24 particles per 1 million BMMSCs (n=3 donors) (FIG. 25) were purified. This confirms the reproducible production of a high yield of exosomes from cells from different donors using our protocol. Thus, it was confirmed that the protocols described in this example section can be employed for all cell types listed in the present disclosure.

FIG. 26 depicts the comparison of purity of exosomes purified by three methods (i) single step ultracentrifugation (UC_step1), (ii) s\30% sucrose cushion (iii) iodixanol gradient UC (IDX). (A) Sucrose cushion and iodixanol gradient methods gave comparable purity and low levels of VEGF compared to UC_Step 1 (single step ultracentrifugation) while retaining therapeutic factors such as HGF (B).

### Example 6

Priming of Mesenchymal Stem Cells with Corneal Stem Cell Derived-Conditioned Medium (CSSC-CM)

The CSSC were cultured as per the protocol described in Example 1, and the MSC was cultured as per the protocol described in Example 2.

Priming—The aspect of priming as described in the present disclosure discloses a step of culturing the MSC in a culture medium comprising 5-50% by volume of CSSC-CM till confluency to obtain primed MSC.

Expansion—Once the required confluency is achieved, the primed MSC were expanded by the means of culture methods disclosed in the Examples 3 and 4.

The step of priming can also be done by the methods as disclosed in the Examples 3 and 4. It is contemplated that wherever required in the methods of Examples 3 and 4, the conventional culture medium would be replaced with the culture medium comprising 5-50% by volume of CSSC-CM.

### Results:

Characterization of CSSC-Conditioned Media (CSSC-CM) Primed hBMMSCs in 2D

Un-primed control (without the step of priming by CSSC-CM) BMMSCs and CSSCs were cultured as described in Examples 2 and 1, respectively. BMMSCs were cultured in 10 & 25% CSSC-CM supplemented xenofree media till >90% confluency. Cells were incubated in serum-free media for 24 hours and conditioned media was collected for processing. Secretome of BMMSCs from three independent donors (#200, #227, #257) were harvested alongside CSSCs and CSSC-primed BMMSC (only Donor #200) and secreted levels of VEGF, HGF and IL-6 were quantified. BMMSCs from all three donors were found to secrete more VEGF than CSSCs (FIG. 18A), while CSSCs were observed to express more HGF levels (FIG. 18 B). As shown in FIG. 18, the levels of VEGF were reduced in CSSC-CM primed BMMSCs (Donor #200) in a dose dependent manner, when compared to unprimed BMMSCs (Donor #200) (FIG. 18 C) (10% CSSC-CM priming as compared to 25% CSSC-CM priming). Interestingly, the levels of HGF secreted by CSSC-CM primed BMMSCs were modestly increased when compared to un-primed BMMSCs (FIG. 18A) (dose dependent increase also visible).

CSSCs were found to secrete significantly lower levels of pro-inflammatory IL-6 compared to BMMSCs while priming of BMMSCs with CSSC-CM resulted in a marked decrease in the level of IL-6 secreted by the primed BMMSCs (FIG. 18 B). These data demonstrate that priming BMMSCs with CSSC-CM skews BMMSC to behave more like CSSCs. MSCs from several sources (BM-UC-, AD-, DP, WJ-) are commercially supplied at clinical grade while CSSCs are not made commercially available. Hence, priming MSCs with CSSC-CM will be able to reduce the dependence on a continuous supply of fresh donor corneas for the production of CSSCs and derived exosomes for clinical applications.

Since CSSC—conditioned media contains HGF, BMMSC-CM was spiked with 10% and 25% CSSC-CM prior to assaying. As shown by the clear grey bars, the additive HGF values were quantified in these controls. The controls demonstrate that the priming effects on HGF are not due to the additive effect of CSSC-CM+BMMSC-CM. Nerve Growth factor (NGF) and soluble Fms Related Receptor Tyrosine Kinase 1 (sFLT1) were detected in CSSC secretome while BMMSC-secretome from three donors (ID #200, #227 and #257) did not express detectable levels of the proteins (by western blot). Priming of BMMSC Donor #200 with CSSC-CM induced the secretion of NGF and sFLT1 in the secretome at both 10% and 25% supplementation (FIG. 18 D).

Advantage—Priming hBM-MSCs with CSSC-CM skews the phenotype of BM-MSCs towards a more CSSC-like profile. This will help in circumventing the need to isolate fresh CSSCs from human donor corneas, which are difficult to procure and will also minimize donor to donor variation in exosome batch production. In addition, the yield of CSSCs is also very poor, when compared to commercially available sources of BM-MSCs. Hence, the protocol to reprogram BM-MSCs to behave like CSSCs will provide sufficient cell yields for the production of therapeutic exosomes. Approximately, 0.5-1M stem cells per donor cornea can be expanded to 4-6M in 3 passages. Commercially available BMMSCs can be expanded from 1M to 80-120M in 3 passages. Hence, 20-30 folds higher cell yield is achieved by using BMMSCs versus CSSCs. However, CSSCs (cornea resident MSCs) have shown to be immensely effective in corneal wound healing that cannot be mimicked by the use of BMMSCs. Therefore, the priming of BMMSCs with CS SC-conditioned media reprograms BMMSCs into CSSC-like stem cells. This protocol will help produce 20-60 folds higher CSSC-like BMMSC cell yield and exosomes. While using CSSC-exosomes can help treat 8-10 corneas at a dose of 0.1-0.5 billion exosomes per eye, the priming protocol proposes to treat 20-60×i.e. 200-600 patients from a single donor cornea. Furthermore, by employing the 3D scalable cell culture process as described in the Examples 3 and 4 further amplification of the cell and exosome yield is achieved by an additional 5-10 folds. Hence, it can be inferred that the combination of CSSC-CM priming protocols with 3D expansion methods (as described in Examples 3 and 4) will yield 100-600 folds higher exosomes yield, allowing the treatment of approximately 1000-5000 patients per donor cornea.

### Advantages of the Present Disclosure

The present disclosure discloses process of culturing MSC to obtain expanded MSC and a MSC-CM. Significant advantages include the scalability of the process as described herein along with the fact that the process is a xeno-free process, therefore, the process of the present disclosure gives a viable option of scalability for meeting the commercial requirements and also provides clinical grade end products in terms of MSC-CM. The MSC-CM is further processed to obtain clinical grade exosomes, secretome, and other cello-derived products which can be used for treating a condition selected from the group consisting of corneal disorders, liver fibrosis, and hyper-inflammatory conditions. As per the process disclosed in the present disclosure, high quality exosome yield of approximately 2 billion purified exosomes is obtained from approximately 1 million MSCs grown in 2D format (as per the Example 1 and 2). By culturing cells employing the process of the present disclosure 3D scalable platforms, at least 5-10 folds amplification can be obtained in exosome yield. As per the present disclosure, the exosome yield is scalable without impacting the production costs. Advantage in terms of total proteins, cell viability and quality can be observed in the Table 5 and Table 6.

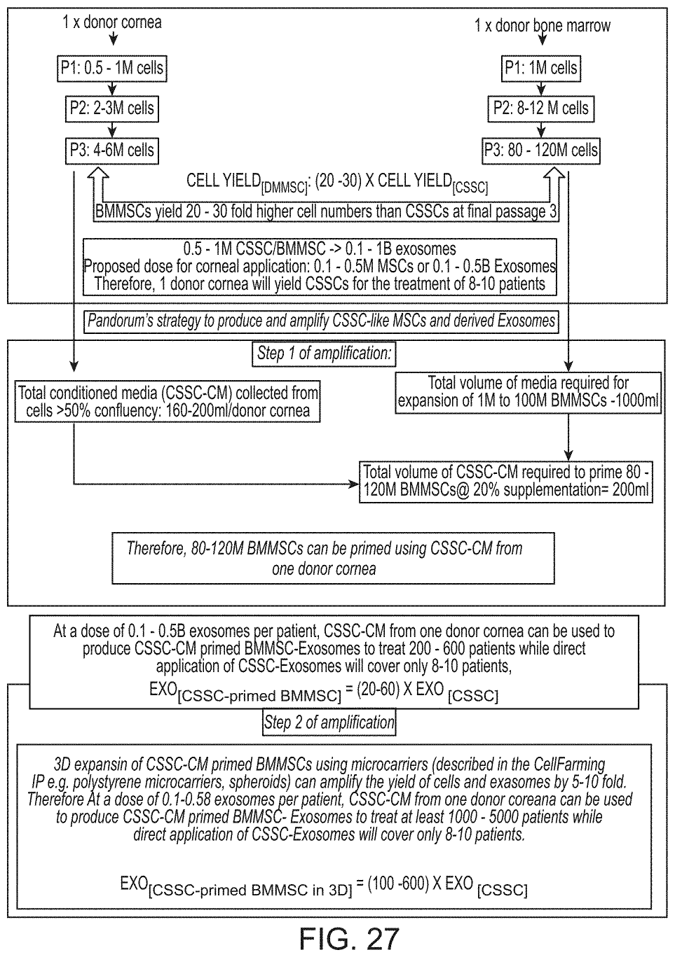

FIG. 27 describes the advantage of priming of the BM-MSC by CSSC-CM (Example 6). The present disclosure provides an advantage that that priming of hBMMSCs with 5-50% (Example 6) CSSC conditioned media reprograms BMMMSCs to a more CSSC-like phenotype as demonstrated by the secretory profile of high HGF and lower VEGF & IL-6 in CSSC-conditioned media primed BMMSCs versus naïve BMMSCs. Therefore, CSSC-CM priming in combination with 3D cell expansion platform can increase the yield of exosomes (and cells) for clinical application by approximately 125 folds i.e. for corneal applications, a dose of 0.5 Billion exosomes from CSSC-CM primed BMMSCs can be administered to approximately 1000 patients to 5000 patients, whereas the direct application of CSSC exosomes will cover only 8-10 patients.

1-3. (canceled)

4. A process for culturing stem cells to obtain a population of expanded stem cells, said process comprising:
a) obtaining a population of stem cells;
b) obtaining microcarriers comprising crosslinked alginate core and crosslinked gelatin surface;
c) suspending the microcarriers in a culture medium, to obtain a suspension;
d) seeding the suspension with the population of stem cells of step (a);
e) culturing the stem cells of step (d) in a culture medium to obtain a population of expanded stem cells adhered to the microcarriers, and a stem cell derived-conditioned medium; and
f) dissolving the microcarriers of step (e) by contacting the microcarriers with a dissolution buffer comprising sodium chloride and trisodium citrate, to obtain a population of expanded stem cells.

5. The process as claimed in claim 4, wherein the microcarriers are in a size ranging from 50-500 μm.

6. The process as claimed in claim 4, wherein the microcarriers comprise sodium alginate in the concentration range of 0.01-20% w/v, and gelatin in the concentration range of 0.1-20% w/v.

7. (canceled)

8. The process as claimed in claim 4, wherein culturing the stem cells of step (d) is done in a culture medium comprising a corneal stromal stem cell derived-conditioned medium, and wherein the corneal stromal stem cell derived-conditioned medium is obtained by culturing of corneal limbal stem cells.

9. (canceled)

10. A process for culturing stem cells, to obtain a population of expanded stem cells, said process comprising:
a) obtaining a population of stem cells;
b) pelleting the stem cells of step (a), to obtain a stem cell pellet;
c) resuspending the stem cell pellet in a culture medium comprising basal medium, to obtain a stem cell suspension;
d) obtaining stem cell spheroids from the stem cell suspension obtained in step (c), wherein the stem cell spheroids are in a range of 600-10,000 cells per spheroid; and
e) culturing the stem cell spheroids of step (d) in a culture medium to obtain a population of expanded stem cells, and a stem cell derived-conditioned medium.

11. The process as claimed in claim 10, wherein the culture medium of step (c) and step (e) comprises methyl cellulose in a concentration range of 0.2-2% with respect to the culture medium.

12-14. (canceled)

15. The process as claimed in claim 10, wherein the culturing of spheroids of step (e) is done in a culture medium comprising corneal stromal stem cell derived-conditioned medium, and wherein the corneal stromal stem cell derived-conditioned medium is obtained from culturing of corneal limbal stem cells.

16. (canceled)

17. The process as claimed in claim 4 or claim 10, wherein the population of stem cells is selected from the group consisting of human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, induced pluripotent stem cells, and corneal limbal stem cells.

18. A stem cell derived-conditioned medium obtained by the process as claimed in claim 4 or claim 10.

19. An expanded stem cell population obtained by the process as claimed in claim 4 or claim 10.

20-21. (canceled)

22. A process for isolating and culturing corneal limbal stem cells, to obtain an expanded corneal stromal stem cell population, said process comprising:
a) obtaining a limbal ring tissue from a human donor cornea;
b) mincing the tissue, to obtain tissue fragments;
c) suspending the fragments in an incomplete medium, to obtain a suspension;
d) subjecting the fragments to digestion in the presence of at least one type of collagenase enzyme at a concentration range of 5-20 IU/μl with respect to the suspension, to obtain digested explants;
e) culturing the digested explants in a complete medium comprising 1-10% human platelet lysate for a period of 10-14 days, to obtain a population of corneal limbal stem cells; and
f) passaging the corneal limbal stem cells of step (e) for a period of 10-14 days, to obtain an expanded corneal stromal stem cell population, and a corneal stromal stem cell derived-conditioned medium.

23-30. (canceled)

31. The process as claimed in claim 22, wherein the tissue fragments have a size in a range of 1-2 mm.

32-35. (canceled)

36. An exosome preparation obtained by a process comprising: (a) harvesting the stem cell derived-conditioned medium as claimed in claim 18; (b) centrifuging the secretome, to obtain a pellet; (c) dissolving the pellet in a low serum xeno free medium, to obtain a crude solution; (d) performing density gradient ultracentrifugation with the crude solution, to obtain a fraction comprising exosomes; and (e) purifying the fraction comprising the exosomes by size exclusion chromatography, to obtain an exosome preparation.

37. A composition comprising the exosome preparation as claimed in claim 36.

38. (canceled)

39. A method for treating a condition selected from the group consisting of corneal disorders, liver fibrosis, and hyper-inflammatory conditions, said method comprising administering the composition of claim 37 to a subject for treating the condition.

40-42. (canceled)

43. The method as claimed in 10, wherein obtaining the stem cell spheroids is either done by a static hanging drop method or by spontaneous aggregation of the stem cells.

44-46. (canceled)
